(19) United States
(12) Patent Application Publication
(10) Pub. No.: US 2006/0212125 A1
(43) Pub. Date: Sep. 21, 2006

(54) Bone repairing material using a chondrocyte having the potential for hypertrophy and a scaffold

(75) Inventor: Hiroyuki Okihana, Itabashi-ku (JP)

Correspondence Address: SEED INTELLECTUAL PROPERTY LAW GROUP PLLC, 701 FIFTH AVE, SUITE 6300, SEATTLE, WA, 98104-7092, US

(73) Assignee: PENTAX Corporation, Itabashi-ku (JP)

(21) Appl. No.: 11/378,874

(22) Filed: Mar. 17, 2006

(30) Foreign Application Priority Data
Mar. 7, 2006 (JP) 2006-061931
Mar. 18, 2005 (JP) 2005-080677

(51) Int. Cl. A61F 2/28; C12N 5/08

(52) U.S. Cl. 623/23.63; 435/384

(57) ABSTRACT

The present invention provides a composite material for enhancing or inducing osteogenesis in a biological organism, comprising A) a chondrocyte having the potential for hypertrophy, and B) a scaffold that is biocompatible with the biological organism. The present invention also provides a method for producing a composite material for enhancing or inducing osteogenesis in a biological organism, comprising A) providing a collected chondrocyte having the potential for hypertrophy, and B) culturing the chondrocyte having the potential for hypertrophy on a scaffold that is biocompatible with the biological organism.

### CROSS-REFERENCE TO RELATED APPLICATIONS

The present application claims priority to Japanese Patent Application Nos. 2005-80677 and 2006-61931, filed on Mar. 18, 2005, and Mar. 7, 2006, respectively which are herein incorporated by reference in their entirety.

### TECHNICAL FIELD

The present invention is directed to a material for enhancing and inducing osteogenesis in a biological organism. In particular, the invention is directed to a composite material using a chondrocyte having the potential for hypertrophy and a scaffold, as well as a method of producing and the use thereof.

### BACKGROUND ART

Promotion of osteogenesis is a preferred method to treat many of diseases associated with bone, or damage or deficits of bone. When bone tissue sustains damage such as fracture, osteoblasts, bone generating cells, proliferate and differentiate to regenerate bone. In a mild case of damage, immobilization of the bone at the affected area allows osteoblasts to be activated, thereby the immediate area is repaired. In circumstances wherein osteoblasts cannot be activated effectively, such as in the case of complex fracture or damage in a joint, or damage in combination with osteomyelitis, autologous bone transplantation has been generally considered as a standard method for repairing damage or deficits. When the defective region is too large to repair with autologous bone, artificial bone may be used in partial combination with autologous bone. However, in humans, sources of autologous bone are limited. In addition, supplying autologous bone is accompanied by high costs and pain to the donor. Moreover, the use of autologous bone causes a new deficit in a region which was originally normal and from which the autologous bone is obtained. There is another disadvantage that an additional operation is required to collect bone, wherein the amount of the bone which can be collected is limited.

In the United States, allogeneic bone obtained from c avaders is often used. Whereas, in Japan, the use of cav aderic tissues is unfamiliar and thus they are not used s o often. Although Bone Banks are an alternative way of p roviding autologous bone, so far, the amount autologous b one supplied in this manner is small.

For example, allogeneic bone obtained from cavaders is often used in the United States. However, it causes the problem of frequent transmission of infection.

Thus, various surgical procedures such as the use of artificial bone implants and bone repair materials have been utilized. However, after these surgical procedures, the prognosis for such procedures is not always good and multiple operations are often needed.

Japanese Laid-Open Patent Publication No. 2003-38635 describes a material for repairing the chondro-osseous defective region, wherein chondrocytes or bone marrow cells are embedded in solubilized atelocollagen and subsequently solidified within a porous body of beta-tricalcium phosphate. This publication, however, does not describe the use of chondrocytes having the potential for hypertrophy. Moreover, this method needs the cells to be embedded into solubilized atelocollagen and subsequently coagulated.

Japanese Laid-Open Patent Publication No. H10-243996 describes a biomaterial for enhancing the calcification of hard tissue, wherein the main components are calcium phosphate compounds and aggregates of osseous cells. The method is not intended to use chondrocytes having the potential for hypertrophy. Further, in this invention, the osseous cells form aggregates and are not intended to be cultured on a scaffold.

Japanese Laid-Open Patent Publication No. 2004-8634 describes a scaffold consisting of a material made from biodegradable molecules containing inclined calcium phosphate, which is capable of effectively regenerating an interface between hard and soft tissue. In this method, inclined calcium phosphate must be used. The scaffold described in this document is not intended for use with chondrocytes having the potential for hypertrophy.

WO 98/16209 describes a synthetic, poorly crystalline apatite (PCA) calcium phosphate containing a biologically active agent and/or cells. In this method, the calcium phosphate used must be poorly crystalline. This document describes in vivo and in vitro examples of chondrogenesis using a composition of the PCA material inoculated with chondrogenic cells (Examples 28-31). However, these are examples of chondrogenesis rather than osteogenesis. Although this document describes an example of heterotropic osteogenesis (Example 27), it is directed to using bone marrow cells. In this document, chondrocytes having the potential for hypertrophy are not described.

Therefore, conventional artificial bone implants and bone repair materials have a problem in that they are not easy-to-use.

Furthermore, conventional artificial bone implants and bone repair materials also have disadvantages compared to autologous-bone such as poor osteogenic ability, difficulty in generating bone, low rigidity and fragility.

Although the proportion of usage of artificial bone is increasing, for the reasons described above, it remains at 20-30%, and autologous bone is used in the remaining 70-80% instances.

In order to improve the above disadvantages of the artificial bone, attempts to utilize regenerative medicine using the regenerative potential of cells have begun. These attempts have also been applied to the treatment of bone deficit. Stem cells derived from bone marrow are generally used in such regenerative medicine.

It has been shown that chondrocytes having the potential for hypertrophy induce osteogenesis when they are pelleted and implanted (Okihana H. and Shimomura Y., Bone 13, 387-393 (1992)). In general, if cells are implanted without pelleting, they disperse and cannot generate bone, thus such cells cannot adequately treat a defective region of bone. However, if they are pelleted, it is difficult to achieve a size suitable for the actual treatment of bone deficit. Conventionally, bone repair using bone marrow cells, mesenchymal stem cells, osteoblasts, which have been utilized in regenerative medicine, is still inferior to that using autologous bone and is not acceptable for conventional use (see, e.g., WO 97/40137, WO 96/23059, Japanese Laid-Open Patent Publication No. 2003-199815, Japanese Laid-Open Patent Publication No. 2003-52365,U.S. Pat. No. 5,486,359, U.S. Pat. No. 5,226,914, WO97/40137, WO99/46366, Ohguchi, H. et al., Acta Orthop. Scand., 60: 334-339 (1989), Caplan, A. I.: J. Orthop. Res., 9: 641-650 (1991), Bruder, S. et al.:. J. Bone Joint Surg., 80A: 985-996 (1998), Yoshikawa, T. et al.: Biomed Material Eng., 8:311-320(1998), Pittenger, M. F. et al.: Science, 284: 143-147 (1999), Bianco, P. & Robey, P. G.: Nature, 414: 118-121 (2001), Quarto, R. et al.: New Eng. J. Med., 344: 385-386 (2001), and Okihana: Medical Science Digest vol. 30 (1) (2004)).

Einhorn, T. A. et al., J. Bone Joint Surg., 66A: 274-279 (1984) describes that the transplantation of allogenic bone into a defective region of bone in rat leads to osteogenesis. However, transplantation of allogenic bone to treat the defective region of the bone in human has many restrictions and is thus unrealistic. Therefore, alternative methods to allogenic bone transplantation are needed.

Bruder, S. P. et al., J. Bone Joint Surg., 80A: 985-996 (1988) describes an experiment wherein a ceramic implant and mesenchymal stem cells are implanted into a defective region of a femur in a dog. This article reports that the transplantation of a ceramic implant and mesenchymal stem cells into a defective region of bone leads to bone adhesion. However, the success rate of the transplantation of ceramic implant and mesenchymal stem cells into a defective region of bone is not as high as autologous bone, and thus this method is not considered to be practical in view of its culture period or cost.

Mesenchymal stem cells in the bone marrow are members of the cell-lineage which differentiates to bone, and have been experimentally used in combination with scaffold for transplantation into a bone deficit region (Bruder, S. P. et al. (1988), supra). Also, it is believed that osteoblasts or precursor cells thereof (such as mesenchymal stem cells) are essential for good osteogenesis. However, while the attempt to transplant a scaffold for osteogenesis and osteoblasts together into a bone deficit region to induce osteogenesis has described (WO 98/16209), there are no descriptions of any practical application of the technique, suggesting that it is not practical technique, similar to the use of mesenchymal stem cells.

I indicated that osteogenesis can be induced by the growth cartilage cells, i.e. a cell other than osteoblasts or precursor cells. However, there are no reports of the use of growth cartilage cells in combination with a scaffold. Thus, it is not possible to estimate the level of osteogenesis attained by using chondrocytes having the potential for hypertrophy with a scaffold. Since osteogenesis is generally induced by osteoblasts, it has not been believed to be realistic to use cells other than osteoblasts.

### SUMMARY OF THE INVENTION

The object of the present invention is to provide a composite material comparative to autologous bone, or at least to allogenic bone, as well as a method for the production and use thereof, which is available to treat large-scale deficits of bone, bone tumors, complex fractures and the like, in a biological organism.

The object of the present invention is to provide a composite material, which is more useful than conventional artificial bone implants and bone repair materials with respect to the rate of bone regeneration, strength of the regenerated bone and the like.

The object of the present invention is to provide a composite material which may be used to improve osteogenesis in a defective region of bone having a size that is incapable of being repaired by fixation alone.

The objects mentioned above have been partially solved in the present invention by finding that a composite material comprising a chondrocyte having the potential for hypertrophy and a biocompatible scaffold that is biocompatible with a biological organism, has a property that causes the unexpected progression of osteogenesis as a combination of cell and a scaffold. Particularly, I found that the combination of a chondrocyte having the potential for hypertrophy and a biocompatible scaffold shows unexpectedly higher rates of osteogenesis than the combination of an osteoblast and a scaffold which has been believed to be essential for osteogenesis, and that the combination of scaffold and cells which was conventionally not considered to be practical can be used to treat bone deficit at a practical level. Considering that until now, according to the common knowledge within the art, osteogenesis is performed by osteoblasts, and that it is thus not practical to use cells other than osteoblasts to promote osteogenesis, the rate of osteogenesis achieved by the present invention is significantly excellent.

The present invention also provides a method for producing and using a composite material comparative to autologous bone, or at least to allogenic bone.

To achieve the objects mentioned above, the present invention provides the following:

In one aspect, the present invention provides a composite material for enhancing or inducing osteogenesis in a biological organism, comprising:

A) a chondrocyte having the potential for hypertrophy, and

B) a scaffold that is biocompatible with the biological organism.

In one embodiment, the present invention provides a composite material for enhancing or inducing osteogenesis in a biological organism, wherein the osteogenesis is for repairing a defective region of bone.

In another embodiment, the composite material according to the present invention is used to ameliorate osteogenesis in a defective region of the bone having a size that is incapable of being repaired by fixation alone.

In one embodiment, the chondrocyte having the potential for hypertrophy employed in the present invention is contained in a region which is selected from the group consisting of a surface, and a region within an internal pore, of the scaffold that is biocompatible with the biological organism.

In another embodiment, the chondrocyte having the potential for hypertrophy employed in the present invention expresses at least one marker selected from the group consisting of type X collagen, alkaline phosphatase, osteonectin, type II collagen, cartilage proteoglycan or components thereof, hyaluronic acid, type IX collagen, type XI collagen and chondromodulin.

In a further embodiment, the chondrocyte having the potential for hypertrophy employed in the present invention is characterized by morphological hypertrophy.

In another embodiment, the chondrocyte employed by the present invention is determined to have the potential for hypertrophy by observing its significant proliferation by preparing a pellet of the cells by centrifugation of 5×10⁵ cells in culture medium, culturing the pellet for a pre-determined period, and comparing a size of the cells observed under a microscope before culture with that after culture.

In one embodiment, the chondrocyte having the potential for hypertrophy employed in the present invention is derived from a mammal.

In another embodiment, the chondrocyte having the potential for hypertrophy employed in the present invention is derived from a human, a mouse, a rat, a rabbit, a dog, a cat, or a horse.

In an alternative embodiment, the chondrocyte having the potential for hypertrophy employed by the present invention is derived from an allogenic individual.

In another embodiment, the chondrocyte having the potential for hypertrophy employed by the present invention is derived from a heterologous individual.

In one embodiment, the chondrocyte having the potential for hypertrophy employed by the present invention is a cell obtained from a portion selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture and the cartilaginous part of bone proliferation phase.

In a further embodiment of the present invention, the epiphysial line of the long bone is a region selected from the group consisting of femoris, tibia, fibula, humerus, ulna and radius.

In a another embodiment of the present invention, the zone of proliferating cartilage of ossicle is a region selected from the group consisting of hand bones, foot bones and sterna. In one embodiment, the chondrocyte having the potential for hypertrophy employed by the present invention is adjusted to a cell density of 1×10⁷ cells/ml to 1×10⁴ cells/ml.

In one embodiment, the chondrocyte having the potential for hypertrophy employed by the present invention is cultured in a medium comprising one selected from the group consisting of Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb), and a combination thereof.

In a further embodiment, the medium employed by the present invention includes a material that enhances the proliferation, differentiation or both of cells.

In another embodiment, the medium employed by the present invention includes at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF).

In one embodiment, the scaffold that is biocompatible with the biological organism employed by the present invention comprises a material selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon, and a combination thereof.

In a further embodiment, the scaffold that is biocompatible with the biological organism employed by the present invention is comprised of calcium phosphate, gelatin, collagen or a combination thereof.

In another embodiment, the scaffold that is biocompatible with the biological organism employed by the present invention is comprised of hydroxyapatite.

In one aspect, the present invention provides a method of producing of a composite material for enhancing or inducing osteogenesis in a biological organism, comprising the steps of:

A) providing a chondrocyte having the potential for hypertrophy, and

B) culturing the chondrocyte having the potential for hypertrophy on a scaffold that is biocompatible with the biological organism.

In one embodiment, step A) of the method of producing a composite material for enhancing or inducing osteogenesis in a biological organism according to the present invention, comprises providing the chondrocyte having the potential for hypertrophy, wherein the potential for hypertrophy is indentifed by the expression of at least one selected from, but not limited to the group consisting of type X collagen, alkaline phosphatase, osteonectin, type II collagen, cartilage proteoglycan or components thereof, hyaluronic acid, type IX collagen, type XI collagen, and chondromodulin, as a marker.

In another embodiment, step A)of the method of producing a composite material for enhancing or inducing osteogenesis in a biological organism according to the present invention, comprises the steps of:

providing the chondrocyte having the potential for hypertrophy, wherein the potential for hypertrophy is indentifed using its hypertrophy as a marker;

preparing a pellet of the cells by centrifugation of 5×10⁵ cells in culture medium;

culturing the pellet for a pre-determined period;

comparing the size of the cells observed under a microscope before culture with that after culture; and

determining the chondrocyte as having the potential for hypertrophy when significant proliferation is observed.

In one embodiment, the chondrocyte having the potential for hypertrophy employed by methods according to the present invention, is derived from a mammal.

In another embodiment, the chondrocyte having the potential for hypertrophy employed by methods according to the present invention, is derived from a human, a mouse, a rat, a rabbit, a dog, a cat or a horse.

In one embodiment, the chondrocyte having the potential for hypertrophy employed by methods according to the present invention, is a cell obtained from a portion selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture and the cartilaginous part of bone proliferation phase.

In a further embodiment of the present invention, the epiphysial line of the long bone is a region selected from the group consisting of femoris, tibia, fibula, humerus, ulna and radius.

In another embodiment of the present invention, the zone of proliferating cartilage of ossicle-is a region selected from the group consisting of hand bone, foot bone and the sterna.

In one embodiment, the chondrocyte having the potential for hypertrophy employed by methods according to the present invention, is adjusted to a cell density of 1×10⁷ cells/ml to 1×10⁴ cells/ml.

In one embodiment, step B) of the method of producing a composite material for enhancing or inducing osteogenesis in a biological organism according to the present invention, comprises culturing the chondrocyte having the potential for hypertrophy in a medium comprising one selected from the group consisting of Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb) and a combination thereof.

In another embodiment, step B) of the method of producing a composite material for enhancing or inducing osteogenesis in a biological organism according to the present invention, comprises culturing the chondrocyte having the potential for hypertrophy in a medium including a substance that enhances the proliferation, differentiation or both of cells.

In one embodiment, step B) of the method of producing a composite material for enhancing or inducing osteogenesis in a biological organism according to the present invention, comprises culturing the chondrocyte having the potential for hypertrophy in a medium including at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF).

In one embodiment, the scaffold that is biocompatibile with the biological organism employed by methods according to the present invention, includes a material selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon and a combination thereof.

In a further embodiment, the scaffold that is biocompatible with the biological organism employed by methods according to the present invention, is calcium phosphate, gelatin or collagen.

In an alternative embodiment, the scaffold that is biocompatible with the biological organism employed by methods according to the present invention, is hydroxyapatite.

In one embodiment, the chondrocyte having the potential for hypertrophy employed by methods according to the present invention, is a cell cultured in a region selected from the group consisting of a surface and within an internal pore of the scaffold that is biocompatible with the biological organism, at 37° C. in the presence of 5-10% CO₂.

In further embodiment, the chondrocyte having the potential for hypertrophy employed by methods according to the present invention, is cultured for a sufficient period such that the chondrocyte having the potential for hypertrophy is fixed on the scaffold that is biocompatible with the biological organism.

In one aspect, the present invention provides for the use of a composite material to produce an implant or a bone repairing material for enhancing or inducing osteogenesis in a biological organism, the composite material comprising:

A) a chondrocyte having the potential for hypertrophy, and

B) a scaffold that is biocompatible with the biological organism.

In one embodiment, the present invention provides a method of repairing a defective region of bone, comprising implanting a composite material including a chondrocyte having the potential for hypertrophy and a scaffold that is biocompatible a biological organism into the defective region of the bone.

In a further embodiment, composite material according to the present invention is used to ameliorate osteogenesis in the defective region of the bone having a size that is incapable of being repaired by fixation alone.

In one aspect, the present invention provides a method of preparing of chondrocyte having the potential for hypertrophy, comprising the steps of obtaining cells from the processus xiphoideus junction located in the inferior portion of the corpus sterni.

### EFFECT OF THE INVENTION

the present invention provides a composite material comparative to autologous bone as well as a method for the production and use thereof, which is available to treat large-scale deficits of bone, bone tumors, complex fractures and the like in a biological organism. Such a composite material can repair bone deficits of a size that is difficult to repair using prior art composite materials, by virtue of its unexpected efficacy in promoting osteogenesis, leading to regeneration of the bone, whereby making it possible to treat regions having a poor prognosis after implantation of prior-art artificial materials. The composite material of the invention includes a biocompatible scaffold and functions in actual implantation therapy. Such a composite material has not been provided by the prior art, and instead is provided by the present invention for the first time.

These and other advantages of the present invention will be apparent from the drawings and a reading of the detailed description thereof.

### BRIEF DESCRIPTION OF THE DRAWINGS

FIG. 1A depicts the processus xiphoideus junction located in the inferior portion of the corpus sterni.

FIG. 1B depicts the growth cartilage layer (center), the corpus sterni consisting of bone (right), and the layer containing chondrocytes without the potential for hypertrophy (left).

FIG. 1C shows a micrograph of chondrocytes having the potential for hypertrophy (growth cartilage cells derived from costa/costal cartilage) one week after culturing a pellet of 5×10⁵ cells, stained with hemotoxylin eosin (HE). Hypertrophy of the cells can be observed compared to FIG. 8. Bar=30 μm.

FIGS. 2A-D show chondrocytes having the potential for hypertrophy diluted in a cell suspension, inoculated to hydroxyapatite, and stained with alkaline phosphatase. The cells (1×10⁶ cells/ml) were inoculated to hydroxyapatite, incubated in a 5% CO₂ incubator at 37° C. for 3 hours (A), 1 day (B), 3 days (C) and 1 week (D), and stained with alkaline phosphatase. Cells were stained red with alkaline phosphatase.

FIG. 2E shows the result of toluidine blue staining of samples stained with alkaline phosphatase in FIG. 2A. With toluidine blue, the same areas as FIG. 2A were stained blue, indicating the presence of cells. The lower part of FIG. 2E is a sectional view of the hydroxyapatite stained with toluidine blue.

FIGS. 2F-H show the result of toluidine blue staining of the samples stained with alkaline phosphatase in FIGS. 2B-D. With toluidine blue, the same areas as FIGS. 2B-D were stained blue, indicating the presence of cells.

FIG. 3A shows HE staining of implanted regions in rats 4 weeks after the subcutaneous implantation of a composite material using chondrocytes having the potential for hypertrophy derived from costa/costal cartilage and gelatin as a biocompatible scaffold. Bar=100 μm.

FIG. 3B shows HE staining of implanted regions in rats 4 weeks after the subcutaneous implantation of a composite material using chondrocytes having the potential for hypertrophy derived from costa/costal cartilage and collagen as a biocompatible scaffold. Bar=100 μm.

FIG. 3C shows HE staining of implanted regions in rats 4 weeks after the subcutaneous implantation of a composite material using chondrocytes having the potential for hypertrophy derived from costa/costal cartilage and hydroxyapatite as a biocompatible scaffold. Bar=200 μm.

FIG. 4 shows HE staining 4 weeks after the subcutaneous implantation of hydroxyapatite alone in rats.

FIG. 5A shows chondrocytes having the potential for hypertrophy derived from sterna diluted in cell suspension, inoculated to hydroxyapatite, and stained with alkaline phosphatase. The cells (1×10 cells/ml) were inoculated to hydroxyapatite, incubated in 5% CO₂ incubator at 37° C. for one week, and stained with alkaline phosphatase. Cells were stained red with alkaline phosphatase.

FIG. 5B shows chondrocytes obtained from regions other than the growth cartilage region of the sterna diluted in cell suspension, inoculated to hydroxyapatite, and stained with alkaline phosphatase. 1×10⁶ cells/ml were inoculated to hydroxyapatite, incubated in 5% CO₂ incubator at 37° C. for one week, and stained with alkaline phosphatase. Cells were not stained with alkaline phosphatase.

FIG. 6A shows chondrocytes the potential for hypertrophy derived from articular cartilage diluted in cell suspension, inoculated to hydroxyapatite, and stained with alkaline phosphatase. 1×10⁶ cells/ml were inoculated to hydroxyapatite, incubated in a 5% CO₂ incubator at 37° C. for one week, and stained with alkaline phosphatase. Cells were not stained with alkaline phosphatase.

FIG. 6B shows the result of toluidine blue staining of samples stained with alkaline phosphatase in FIG. 6A. With toluidine blue, the same areas as FIG. 6A were stained blue, indicating the presence of cells.

FIGS. 7A-D show resting cartilage cells without the potential for hypertrophy diluted in cell suspension, inoculated to hydroxyapatite, and stained with alkaline phosphatase. 1×10⁶ cells/ml were inoculated to hydroxyapatite, incubated in 5% CO₂ incubator at 37° C. for 3 hours (A), 1 day (B), 3 days (C) and 1 week (D), and stained with alkaline phosphatase. The cells were not stained with alkaline phosphatase.

FIGS. 7E-H show the result of toluidine blue staining the samples stained with alkaline phosphatase in FIGS. 7A-D. With toluidine blue, the samples stained blue, indicating the presence of cells.

FIG. 8 shows a micrograph of chondrocytes without the potential for hypertrophy (resting cartilage cells derived from costal cartilage), one week after culturing a pellet of 5×10⁵ cells, stained with HE. Hypertrophy of the cells was not observed compared to FIG. 1. Bar=30 μm.

FIG. 9 shows the rate of osteogenesis observed after implantation of a composite material using chondrocytes having the potential for hypertrophy and hydroxyapatite, a composite material using chondrocytes without the potential for hypertrophy and hydroxyapatite, or hydroxyapatite alone, into a bone deficient region. Empty: the rate of osteogenesis 4 weeks after implantation of hydroxyapatite alone into a bone deficient region in rats' skulls, GC: the rate of osteogenesis 4 weeks after implantation of a composite material using chondrocytes having the potential for hypertrophy (growth cartilage cell obtained from costa/costal cartilage)and hydroxyapatite into a bone deficient region in rats' skulls, RC: the rate of osteogenesis after implantation of a composite material using chondrocytes without the potential for hypertrophy (resting cartilage cells obtained from costal cartilage) and hydroxyapatite into a bone deficient region in rats' skulls.

FIG. 10 shows the quantity of osteogenesis observed after implantation of a composite material using chondrocytes having the potential for hypertrophy and hydroxyapatite, a composite material using chondrocytes without the potential for hypertrophy and hydroxyapatite, or hydroxyapatite alone into a bone deficient region. Empty: the quantity (volume) of osteogenesis 4 weeks after implantation of hydroxyapatite alone into a bone deficient region in rats' skulls, GC: the quantity (volume) of osteogenesis 4 weeks after implantation of a composite material using chondrocytes having the potential for hypertrophy (growth cartilage cell obtained from costa/costal cartilage) and hydroxyapatite into a bone deficient region in rats' skulls, RC: the quantity (volume) of osteogenesis 4 weeks after implantation of a composite material using chondrocytes without the potential for hypertrophy (resting cartilage cells obtained from costal cartilage) and hydroxyapatite into a bone-deficient region in rats' skulls.

### BEST MODE FOR CARRYING OUT THE INVENTION

The present invention is described hereafter. It is to be understood that, unless otherwise described, singular representations throughout the present specification include the concept of plural thereof. Therefore, it is to be understood that, unless particularly described, singular articles such as “a”, “an” and “the” in the English language, “un”, “une”, “le” and “la” in the French language, and “ein”, “eine”, “der”, “die” and “das”, and the like, in the German language, or others include the concept of plural. It should be also understood that terms as used herein have the definitions ordinarily used in the art unless otherwise mentioned. Therefore, unless otherwise defined, all technical and scientific terms used herein have the same meaning as that commonly understood by those skilled in the art. Otherwise, the present application (including definitions) takes precedence.

(Definition of Terms)

The definitions of the terms particularly used herein are listed below.

“Composite material” as used herein refers to a material including a cell and a scaffold.

“Enhancing osteogenesis” as used herein refers to increasing the rate of osteogenesis at a site where osteogenesis has already occurred.

As used herein, “Inducing” osteogenesis refers to causing osteogenesis at a site where the osteogenesis has not occurred.

“Bone deficit” as used herein comprises, but is not limited to: lesions such as bone tumors, osteoporosis, rheumatoid arthritis, osteoarthritis, osteomyelitis, and osteonecrosis; correction such as immobilization of the bone, foraminotomy and osteotomy; trauma such as complex fracture; and bone deficits derived from collecting ilium.

As used herein, “repairing” bone deficit regions refers to making the defective region normal or close to normal.

“Size that is incapable of being repaired by fixation alone” as used herein refers to a lesion of a size requiring the use of implants or bone repairing materials to repair.

“Growth cartilage cell” or “growth chondrocyte” is interchangeably used herein to refer to a cell in a tissue which forms in bone during developmental or growth stages, and periods of bone recovery or proliferation (i.e., growth cartilage). Growth cartilage cell generally refers to a tissue which forms bone during growth stage, while it herein means a tissue which forms bone during developmental or growth stages, and periods of bone recovery or proliferation. The growth cartilage cell is also referred to as hypertrophic cartilage, calcified cartilage or epiphysial (line) cartilage. When using growth cartilage cell in humans, cells derived from a human are preferred, but it is also possible to use non-human cells since problems such as immunological rejection can be avoided using techniques well-known in the art.

The growth cartilage cell according to the present invention is derived from a mammal, preferably from a human, a mouse, a rat, a rabbit, a dog, a cat or a horse.

The growth cartilage cell according to the present invention can be sampled from the chondro-osseous junction of costa, epiphysial line of long bone (e.g., femoris, tibia, fibula, humerus, ulna, and radius), epiphysial line of vertebra, zone of proliferating cartilage of hand bone, foot bone, breast bone and others, perichondrium, bone primordium formed from cartilage of fetus, the callus region of a healing bone-fracture, and the cartilaginous part of bone proliferation phase. These chondrocytes can be prepared, for example, by the methods described in the Examples of the present specification.

“A chondrocyte having the potential for hypertrophy” as used herein refers to a cell which can undergo hypertrophic growth in the future. A chondrocyte having the potential for hypertrophy includes a “growth cartilage cell” collected from living organism directly, as well as any other cells having the potential for hypertrophy determined by a method for determining “the potential for hypertrophy” defined hereinafter.

The chondrocyte having the potential for hypertrophy according to the present invention is typically derived from a mammal, preferably a human, a mouse., a rat, a rabbit, a dog, a cat or a horse. When using chondrocytes having the potential for hypertrophy in humans, the cells are preferably derived from a human, but it is also possible to use non-human cells since problems such as immunological rejection can be avoided using techniques well-known in the art. The chondrocyte having the potential for hypertrophy according to the present invention can be obtained, for example, from the chondro-osseous junction of costa, epiphysial line of long bone (e. g., femoris, tibia, fibula, humerus, ulna, and radius), epiphysial line of vertebra, zone of proliferating cartilage of e. g., hand bones, foot bones, sterna, perichondrium, bone primordium formed from cartilage of fetus, the callus region of a healing bone-fracture, and the cartilaginous part of bone proliferation phase. The chondrocyte having the potential for hypertrophy according to the present invention can be obtained by inducing the differentiation of an undifferentiated cell.

The chondrocyte having the potential for hypertrophy may be morphologically characterized by hypertrophy.

“Hypertrophy” as used herein can be determined morphologically under a microscope. As used herein, hypertrophic cell refers to a cell observed adjacent to the growth layer, which aligns in a columnar state, or alternatively refers to a cell that is larger than the surrounding cells.

Cells are determined to have the potential for hypertrophy if significant proliferation is observed by preparing a pellet of the cells by centrifugation of 5×10⁵ cells in culture medium, culturing the pellet for a pre-determined period, and comparing the size of the cells observed under a microscope before and after culture.

“Resting cartilage cell” and “resting cartilage cell” are interchangeably used herein to refer to a cartilage cell or chondrocyte located in the region apart from the chondro-osseous junction of the costa, which is a tissue that exists as cartilage throughout the entire lifetime. A cell located in the resting cartilage is referred to as a resting cartilage cell.

“Articular cartilage cell” as used herein refers to a cell in cartilaginous tissue (articular cartilage) located on an articular surface.

The chondrocyte as used herein is determined by identifying the expression of at least one selected from the group consisting of type II collagen, cartilage proteoglycan (aglycan) or components thereof, hyaluronic acid, type IX collagen, type XI collagen, and chondromodulin as a marker. Among chondrocytes, a hypertrophic cell is further determined by identifying the expression of at least one selected from the group consisting of type X collagen, alkaline phosphatase, and osteonectin. Chondrocytes not expressing any of type X collagen, alkaline phosphatase or osteonectin, are determined not to have hypertrophic potency. Therefore, the chondrocyte having the potential for hypertrophy described herein is also determined by identifying the expression of at least one selected from chondrocyte markers and of at least one selected from markers for chondrocytes having the potential for hypertrophy, instead of observing morphological hypertrophy. The localization or expression of these markers is identified by any method of analyzing proteins or RNA extracted from cultured cells, such as specific staining, immunohistochemical methods, in situ hybridization, Western blotting, or PCR.

“Chondrocyte marker” as used herein refers to any substances whose localization or expression in a chondrocyte aids in the identification of the chondrocyte. Preferably, it refers to any substances which can be used to identify the chondrocyte by their localization or expression (for example, localization or expression of type II collagen, cartilage proteoglycan (aglycan) or components thereof, hyaluronic acid, type IX collagen, type XI collagen, or chondromodulin).

“Marker for chondrocyte having the potential for hypertrophy” as used herein refers to any substances whose localization or expression in a chondrocyte having the potential for hypertrophy aids in identification of the chondrocyte. Preferably, it refers to any substance which can be used to identify the chondrocyte having the potential for hypertrophy by their localization or expression (for example, localization or expression of type X collagen, alkaline phosphatase and osteonectin).

“Cartilage proteoglycan” as used herein refers to a macromolecule, wherein plurality of glucosaminoglycans such as chondroitin tetrasulfate, chondroitin hexasulfate, keratan sulfate, O-linked oligosaccharide, N-linked oligosaccharide and others, combine with a core protein. The cartilage proteoglycan further binds to hyaluronic acid via a linkage protein to form cartilage proteoglycan aggregate. In cartilaginous tissue, the glucosaminoglycan is rich and occupies 20-40% of dry weight of the tissue. Cartilage proteoglycan is also referred to as aglycan.

“Bone proteoglycan” as used herein refers to a macromolecule which has a smaller molecular weight than cartilage proteoglycan, wherein glucosaminoglycans such as chondroitin sulfate, dermatan sulfate, O-linked oligosaccharides, N-linked oligosaccharides and others, combine with core protein. In bone tissue, glucosaminoglycan occupies 1% or less of dry weight of decalcifying bone. Bone proteoglycan may include decorin and biglycan.

“Osteoblast” as used herein is a cell which locates on bone matrix, and which forms and calcifies the bone matrix. Osteoblasts are a cell of 20-30μm diameter, and of cubic or columnar form. As used herein, osteoblast may include “preosteoblast”, which is a precursor cell of osteoblasts.

Osteoblasts are determined by the expression of at least one selected from the group consisting of type I collagen, bone proteoglycan (e. g. decorin, biglycan), alkaline phosphatase, osteocalcin, matrix Gla protein, osteoglycin, osteopontin, bone sialic acid protein, osteonectin and pleiotrophin as a marker. Additionally, osteoblasts can be determined by identifying that chondrocyte markers such as (type II collagen, cartilage proteoglycan (aglycan) or components thereof, hyaluronic acid, type IX collagen, type XI collagen, or chondromodulin), are not expressed thereby. These markers are identified by their localization or expression by any methods of analyzing proteins or RNA extracted from cultured cells, such as specific staining, immunohistochemical methods, in situ hybridization, Western blotting, or PCR.

“Osteoblast marker” as used herein refers to any substances whose localization or expression in an osteoblast aids in the identification of the osteoblast. Preferably, it refers to any substances which can be used to identify osteoblasts by their localization or expression (for example, localization or expression of type I collagen, Bone proteoglycan (e. g. decorin, biglycan), alkaline phosphatase, osteocalcin, matrix Gla protein, osteoglycin, osteopontin, bone sialic acid protein, osteonectin or pleiotrophin). Osteoglycin is referred to as osteoinductive factor (OIF). Osteopontin is referred to as BSP-I or 2ar. Bone sialic acid protein is referred to as BSP-II. Pleiotrophin is referred to as osteoblast specific protein (OSF-1). Osteonectin is referred to as SPARC, or BM-40.

Osteoblasts may be identified, for example by:

finding a cell to be positive for a marker that only identifies osteoblasts;

finding a cell to be positive for a marker identifying osteoblasts and chondrocytes having the potential for hypertrophy, while not identifying chondrocytes, and finding said cell to be positive for a marker that identifies osteoblasts and chondrocytes, while not identifying chondrocytes having the potential for hypertrophy;

finding a cell to be positive for a marker identifying osteoblasts and chondrocytes having the potential for hypertrophy, but to be negative for a marker that does not identify osteoblasts, while identifying chondrocytes having the potential for hypertrophy; or

finding a cell to be positive for a marker identifying osteoblasts and chondrocytes as positive, but to be negative for a marker that does not identifies osteoblasts, while identifying chondrocytes.

Chondrocytes having the potential for hypertrophy may be identified, for example by:

finding a cell to be positive for a marker that only identifies chondrocytes having the potential for hypertrophy;

finding a cell to be positive for a marker identifying chondrocytes having the potential for hypertrophy and osteoblasts, while not identifying chondrocytes, and finding said cell to be positive for a marker that identifies chondrocytes having the potential for hypertrophy and chondrocytes, while not identifying osteoblasts;

finding a cell to be positive for a marker identifying chondrocytes having the potential for hypertrophy and osteoblasts, but to be negative for a marker that does not identifies chondrocytes having the potential for hypertrophy, while identifying osteoblasts; or

finding a cell to be positive for a marker identifying chondrocytes having the potential for hypertrophy and chondrocytes, but to be negative for a marker that does not identifies chondrocytes having the potential for hypertrophy, while identifying chondrocytes.

Chondrocytes (without the potential for hypertrophy) may be identified, for example by:

finding a cell to be positive for a marker that only identifies chondrocytes;

finding a cell to be positive for a marker identifying chondrocytes and osteoblasts, while not identifying chondrocytes having the potential for hypertrophy, and finding said cell to be positive for a marker that identifies chondrocytes and chondrocytes having the potential for hypertrophy, while not identifying osteoblasts;

finding a cell to be positive for a marker identifying chondrocytes and osteoblasts, but to be negative for a marker that does not identifies chondrocytes, while identifying osteoblasts; or

finding a cell to be positive for a marker identifying chondrocytes and chondrocytes having the potential for hypertrophy, but to be negative for a marker that does not identifies chondrocytes, while identifying chondrocytes having the potential for hypertrophy.

Chondrocytes, chondrocytes having the potential for hypertrophy and osteoblasts may be identified herein, for example using the combination of markers listed in Table 1 below:

TABLE 1

| | | chondrocyte | |
|---|---|---|---|
| | | having the | |
| | | potential for | |
| | chondrocyte | hypertrophy | osteoblast |
| type II collagen, | ● | ● | — |
| cartilage proteoglycan | | | |
| (aglycan), hyaluronic | | | |
| acid, type IX collagen, | | | |
| type XI collagen, | | | |
| chondromodulin | | | |
| type X collagen | — | ● | — |
| alkaline phosphatase, | — | ● | ● |
| osteonectin | | | |
| type I collagen, bone | — | — | ● |
| proteoglycan (e.g. | | | |
| decorin, biglycan), | | | |
| osteocalcin, matrix Gla | | | |
| protein, osteoglycin, | | | |
| osteopontin, bone sialic | | | |
| acid protein, | | | |
| pleiotrophin | | | |

●: expressed
—: not expressed

“Mesenchymal stem cell” as used herein refers to a stem cell observed in mesenchymal tissue. The mesenchymal tissue includes, but is not limited to bone marrow, adipose tissue, vascular endothelium, smooth muscle, cardiac muscle, skeletal muscle, cartilage, bone, and ligament. Mesenchymal stem cells are typically derived from bone marrow, adipose tissue, synovial tissue, muscular tissue, peripheral blood, placental tissue, menstrual blood, or cord blood.

(Scaffold)

“Scaffold” as used herein refers to a material to support cells. The scaffold has constant strength and biocompatibility. As used herein, the scaffold is produced from biological materials or naturally supplied materials, or naturally occurring materials or synthetically supplied materials. As used herein, the scaffold is formed from materials other than organisms such as tissues or cells (i.e. noncellular material). As used herein, the scaffold is a composition formed from materials other than organisms such as tissues or cells, including materials derived from living organisms such as collagen or hydroxyapatite. As used herein, “organism” refers a material-system organized to have living function. That is, the term organism discriminates living beings from other material-systems. The concept of the organism comprises cells, tissues or others, while materials derived from living being, extracted from the organism, are not included in the organism. The scaffold region to which cells are fixed includes a surface of the scaffold, or an internal pore of the scaffold if it has such internal pore that can contain cells. For example, a scaffold made from hydroxyapatite includes many pores which can normally contain cells sufficiently.

The material for the scaffold includes, but is not limited to a material selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon, and combinations thereof. Preferably, the scaffold materials is calcium phosphate, gelatin, or collagen. More preferably, the scaffold material is hydroxyapatite.

This scaffold may be provided in any form such as a granular form, block form, or sponge form. This scaffold may be porous or non-porous. For such scaffolds, those commercially available (e.g., from PENTAX Corporation, OLYMPUS Corporation, Kyocera Corporation, Mitsubishi Pharma Corporation, Dainippon Sumitomo Pharmaceuticals, Kobayashi Pharmaceuticals Co. Ltd., Zimmer Inc.) can be used. Standard procedures for preparation and characterization of scaffolds are known in the art, which only require routine experimentation and techniques commonly known in the art. For example, see, U.S. Pat. No. 4,975,526; U.S. Pat. No. 5,011,691; U.S. Pat. No. 5,171,574; U.S. Pat. No. 5,266,683; U.S. Pat. No. 5,354,557; and U.S. Pat. No. 5,468,845, which are incorporated herein by reference. Other scaffolds are also described, for example, in the following documents: articles for biocompatible materials, such as LeGeros and Daculsi, Handbook of Bioactive Ceramics, II pp. 17-28 (1990, CRC Press); other published descriptions, such as Yang Cao, Jie Weng, Biomaterials 17 (1996) pp. 419-424; LeGeros, Adv. Dent. Res. 2, 164 (1988); Johnson et al., J. Orthopaedic Research, 1996, vol. 14, pp. 351-369; and Piattelli et al., Biomaterials 1996, vol. 17, pp. 1767-1770, the disclosures of which are herein incorporated by reference.

“Calcium phosphate” as used herein is the generic name for phosphates of calcium, which include, for example, but are not limited to compounds represented by the following chemical formulas: CaHPO₄, Ca₃(PO₄)₂, Ca₄O(PO₄)₂, Ca₁₀(PO₄)₆(OH)₂, CaP₄O₁₁, Ca(PO₃)₂, Ca₂P₂O₇, or Ca(H₂PO₄)₂.H₂O.

“Hydroxyapatite” as used herein refers to a compound whose general composition is Ca₁₀(PO₄)₆(OH)₂, which is a main component of mammalian hard tissues (bone and teeth), like collagen. Although hydroxyapatite contains a series of calcium phosphates as described above, the PO₄ and OH components within the apatite in the hard tissues of biological organisms are often substituted with a CO₃ component in body fluids. Furthermore, hydroxyapatite is a material that is safety approved by the Ministry of Health, Labour and Welfare of Japan, and the FDA (U. S. Food and Drug Administration). Although many commercially available hydroxyapatites are non-absorbable into the body and remain in the body being hardly absorbed, some are absorbable.

A scaffold that is biocompatible with a biological organism includes, but is not limited to a material selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon, and a combination thereof.

“Collagen” as used herein has similar meaning to that commonly used in the art in the broadest sense. It is a main component of the extracellular matrices of animals. Collagen is available from e. g., Nitta Gelatin Inc., the Japan Institute of Leather Research, Wako Pure Chemical Industries Ltd., Nacalai tesque, Funakoshi Co. Ltd., Sigma-Aldrich and Merck. Collagens from other sources may also be used in the present invention.

“Gelatin” as used herein has similar meaning to that commonly used in the art in the broadest sense. It can be obtained by degrading (such as thermally degrading) collagen collected from skin, tendons or bones of animals. Gelatin is recognized as a water-soluble protein irreversibly converted as a result of cleavage of the ionic bonding or hydrogen bonding between peptide chains of collagen. Gelatin can be available from e. g., Nitta Gelatin Inc., Japan Institute of Leather Research, Wako Pure Chemical Industries Ltd., Nacalai tesque, Funakoshi Co. Ltd., Sigma-Aldrich and Merck. Gelatins from other sources may also be used in the present invention.

“Biocompatible” as used herein refers to compatibility with the tissues or organs of biological organisms without evoking toxicity, immune responses, damage or other adverse effects. Biocompatible materials which can be used in the present invention include, but are not limited to calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon, and a combination thereof.

“Cellular physiologically active substance” or “physiologically active substance” are interchangeably used herein to refer to a substance which affects cells or tissues. Such effects comprise, for example, but are not limited to the control or modification of the cells or tissues. The physiologically active substance includes cytokines or growth factors. The physiologically active substance may be a naturally occurring or synthesized substance. Preferably, the physiologically active substance is produced in a cell. It also includes substances produced in a cell, or substances having a function similar to, but modified from, those produced in a cell. In the present invention, the physiologically active substance may be in the form of protein including peptides, or in the form of nucleic acids, or in other forms.

“Cytokine” as used herein is defined as having a similar meaning to that used in the art in the broadest sense. It refers to a physiologically active substance produced in a cell that affects the same or a different cell. Generally, a cytokine is a protein or polypeptide, and has activities that control the immune response, modulate the endocrine system, modulate the nervous system, effect anti-tumor action, effect anti-viral action, modulate cell growth, modulate cell differentiation, modulate cellular function, and others. In the present invention, cytokines may be in form of protein or nucleic acids. However at the time of actually affecting cells, cytokines are often in form of protein, including peptides.

“Growth factor” or “cellular growth factor” as used interchangeably herein, refer to a substance which enhances or controls the induction of the growth and differentiation of cells. Growth factor is also a proliferation or development factor. In cell culture or tissue culture, growth factors can be added to the medium and substituted for the function of macromolecules in serum. It is proved that, in addition to cell growth, many growth factors function as factors that regulate differentiation.

Cytokines associated with osteogenesis typically include factors such as transforming growth factor-beta (TGF-beta), bone morphogenetic factor (BMP), leukemia inhibitory factor (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF); and compounds such as ascorbic acid, dexamethasone, and glycerophosphoric acid.

Since physiologically active substances such as cytokines and growth factors generally have redundancy, cytokines or growth factors known by another name and function (such as cell adhesion activity or cell-matrix adhesion activity) can be also used in the present invention, as long as they have the activity of the physiologically active substance used in the invention. Cytokines or growth factors can be used in the implementation of the invention, as long as they have preferred activity (such as stem cell growth activity or osteoblast differentiation activity) for the present invention.

“Derived from syngenic” as used herein means derived from an autologous, pure line, or inbred line.

“Derived from an allogenic individual” as used herein means derived from another individual of the same species that is genetically different.

“Derived from a heterologous individual” as used herein means derived from a heterologous individual. Thus, for example, when recipient is human, cells from rat are “deriving from an individual who is heterologous in relation to a biological organism”.

“Subject” as used herein refers to a biological organism to which a treatment of the present invention is applied. It is also referred to as a “patient”. The subject or patient may be a human, a mouse, a rat, a rabbit, a dog, a cat or a horse, preferably a human.

“Implant” or “bone reparing material” as used herein is utilized as having the meaning generally used in the art. As used herein, they are substantially used in the same sense, but as particularly defined, “implant” means all material used to fill, while “bone repairing material” means a material used to repair a defective region of bone.

### Description of the Preferred Embodiments

The best modes of the present invention are described below. It is appreciated that the embodiments provided below are be provided for the purpose of better understanding of the invention and that the scope of the invention should not be limited to the following description. Therefore, it is apparent that those skilled in the art can read the descriptions herein and modify them appropriately within the scope of the present invention.

(Composite Material)

In one aspect, the present invention provides a composite material for enhancing or inducing osteogenesis in a biological organism. Conventionally, artificial bone implants or bone repair materials to enhance or induce osteogenesis in a defective region of bone has not attained sufficiently satisfactory results with respect to rate of bone regeneration, strength of the regenerated bone or the like. The effect of the invention is to provide a composite material which can repair bone deficits wherein regeneration efficiency is poor in the prior art, and induce regeneration of bone, thereby making it possible to treat regions wherein implantation therapy with artifacts has been conventionally difficult. The composite material of the present invention also can be used to ameliorate osteogenesis in a defective region of bone having a size that is incapable of being repaired by fixation alone. Such a composite material comprises A) a chondrocyte having the potential for hypertrophy and B) a scaffold that is biocompatible with the biological organism.

In one preferred embodiment, the chondrocyte according to the present invention expresses at least one selected from the group consisting of type X collagen, alkaline phosphatase, osteonectin, type II collagen, cartilage proteoglycan or components thereof, hyaluronic acid, type IX collagen, type XI collagen, and chondromodulin, as a marker. Thus, the chondrocyte having the potential for hypertrophy of the present invention is characterized by morphological hypertrophy, and expresses at least one selected from this marker group. In one preferred embodiment, the chondrocyte having the potential for hypertrophy according to the present invention can be identified by confirming expression of the above chondrocyte markers and examining its morphological hypertrophy under a microscope.

In another embodiment, the chondrocyte having the potential for hypertrophy according to the present invention is derived from a mammal, preferably a human, a mouse, a rat, a rabbit, a dog, a cat, or a horse. The chondrocyte having the potential for hypertrophy according to the present invention may be isolated or induced from, for example, a region such as the chondro-osseous junction of costa, epiphysial line of long bone (e. g., femoris, tibia, fibula, humerus, ulna, and radius), epiphysial line of vertebra, zone of proliferating cartilage of ossicle (e.g., hand bones, foot bones and sterna), perichondrium, bone primordium formed from cartilage of fetus, the callus region of a healing bone-fracture and the cartilaginous part of bone proliferation phase. The chondrocyte having the potential for hypertrophy according to the present invention can be obtained by inducing the differentiation.

The chondrocyte having the potential for hypertrophy according to the present invention is normally adjusted to a cell density of 1×10⁷ cells/ml to 1×10⁴ cells/ml, however cell densities of less than 1×10⁴ cells/ml or more than 1×10⁷ cells/ml may also be used. When the cell density is less than 1×10⁴ cells/ml, the chondrocyte having the potential for hypertrophy can be proliferated in an incubator. When the cell density is more than 1×10⁷ cells/ml, they can be used without any treatment, but optionally, may be inoculated on a larger scaffold or diluted to an appropriate concentration in culture media. In one embodiment of the present invention, the cell density of the chondrocyte having the potential for hypertrophy can be for instance, 0.5-1×10⁶/cm³ (ml), or 1×10⁵/cm³ (ml). In another embodiment of the present invention, the density of the chondrocyte having the potential for hypertrophy can be 4×10⁴/cm³ (ml).

The cell used in the present invention may be cultured in any medium, which may include, but is not limited to: Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb) or a combination thereof. The chondrocyte having the potential for hypertrophy may be a cell cultured in media containing substances that enhance the proliferation, differentiation or both of the cells, preferably which include, but are not limited to media containing substances such as at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF).

Ham's F12 medium used herein is comprised of, for example, CaCl₂(anhydrate) 33.20 mg/L, CuSO₄.5H₂O 0.0025 mg/L, FeSO₄.7H₂O 0.83 mg/L, KCl 223.60 mg/L, MgCl₂(anhydrate) 57.22 mg/L, NaCl 7599.00 mg/L, NaHCO₃ 1176.00 mg/L, Na₂HPO₄(anhydrate) 142.00 mg/L, ZnSO₄.7H₂O 0.86 mg/L, D-glucose 1802.00 mg/L, hypoxanthine Na 4.77 mg/L, linoleic acid 0.08 mg/L, lipoic acid 0.21 mg/L, phenol red 1.20 mg/L, putrescine 2HCl 0.161 mg/L, sodium pyruvate 110.00 mg/L, thymidine 0.70 mg/L, L-alanine 8.9 mg/L, L-arginine.HCl 211.00 mg/L, L-asparagine.H₂ 015.00 mg/L, L-asparaginic acid 13.00 mg/L, L-cysteineHCl.H₂O 35.00 mg/L, L-glutamate 14.70 mg/L, L-alanyl-L-glutamine 217.00 mg/L, glycine 7.50 mg/L, L-histidine HCl.H₂O 21.00 mg/L, L-isoleucine 4.00 mg/L, L-Leucine 13.00 mg/L, L-lysine HCl 36.50 mg/L, L-methionine 4.50 mg/L, L-phenylalanine 5.00 mg/L, L-proline 34.50 mg/L, L-serine 10.50 mg/L, L-threonine 12.00 mg/L, L-tryptophan 2.00 mg/L, L-tyrosine.2Na.2H₂O 7.80 mg/L, L-valine11.70 mg/L, biotin 0.007 mg/L, D-Ca pantothenate 0.50 mg/L, choline chloride 14.00 mg/L, folic acid 1.30 mg/L, i-inositol 18.00 mg/L, niacinamide 0.04 mg/L, pyridoxine HCl 0.06 mg/L, riboflavin 0.04 mg/L, thiamine HCl 0.30 mg/L, and vitamin B₁₂ 1.40 mg/L).

MEM medium used herein is comprised of, for example, CaCl₂ (anhydrate) 200.00 mg/L, KCl 400.00 mg/L, MgSO₄ (anhydrate) 98.00 mg/L, NaCl 6800.00 mg/L, NaHCO₃ 2200.00 mg/L, NaH₂PO₄ H₂O 140.00 mg/L, D-glucose 1000.00 mg/L, phenol red 10.00 mg/L, L-arginine.HCl 126.00 mg/L, L-cystine.2HCl 31.00 mg/L, L-glutamine 292.00 mg/L, L-histamine HCl.H₂O 42.00 mg/L, L-isoleucine 52.00 mg/L, L-leucine 52.00 mg/L, L-lysine HCl 73.00 mg/L, L-methionine 15.00 mg/L, L-phenylalanine 32.00 mg/L, L-threonine 48.00 mg/L, L-tryptophan 10.00 mg/L, L-tyrosine.2Na.2H₂O 52.00 mg/L, L-valine 46.00 mg/L, D-Ca pantothenate 1.00 mg/L, choline chloride 1.00 mg/L, folic acid 1.00 mg/L, i-inositol 2.00 mg/L, niacinamide 1.00 mg/L, pyridoxal HCl 1.00 mg/L, riboflavin 0.10 mg/L, thiamine HCl 1.00 mg/L.

The scaffold that is biocompatible with a biological organism used in the present invention may be any scaffold as long as it has biocompatibility. Such scaffolds include a material, for example, selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon, and combination thereof. Preferably, the scaffold that is biocompatible with a biological organism is calcium phosphate, gelatin, or collagen, and more preferably hydroxyapatite in various forms (e. g., crystalline hydroxyapatite or uninclined hydroxyapatite). The biocompatibility of the scaffold can be measured with at least one test selected from the group consisting of a intraosteal implantation test, a reverse mutation test, a chromosomal aberration test, a cytotoxicity test, a intramuscular implantation test, a skin sensitization test, a skin or an intradermal irritation test, a pyrogen test, a hemolysis test, an antigenicity test, an acute toxicity test, and a repeat-dose test. Preferably, the compatibility of the scaffold can be measured with all of the tests described above.

A subcutaneous test for osteogenesis is a test for evaluating osteogenic function to generate bone in a region wherein bone does not originally exist, which is also referred to as parosteosis. Since this test can be performed easily, it is broadly used in the art. In the case of bone treatment, a bone deficit test can be used as a method of testing. Osteogenesis is performed by osteoblasts already existing in the immediate environment of a deficit and also those that are induced/migrate thereto. Thus, it is normally believed that the rate of osteogenesis is better in the bone deficit test than in the subcutaneous test. It is well-known that the result of the subcutaneous test is consistent with the rate of osteogenesis in the actual bone deficit (see, e. g., Urist, M. R.: Science, 150: 893-899 (1965), Wozney, J. M. et al.,: Science, 242: 1528-1532 (1988), Johnson, E. E. et al.,: Clin. Orthop., 230: 257-265(1988), Ekelund, A. et al.,: Clin. Orthop., 263: 102-112 (1991), and Riley, E. H. et al.,: Clin. Orthop., 324: 39-46 (1996)). Therefore, if osteogenesis is observed as the result of the subcutaneous test, those skilled in the art understand that osteogenesis should also be induced in the bone defective test.

(Method of Production)

In one aspect, the present invention provides a method for producing a composite material for enhancing or inducing osteogenesis in a biological organism, comprising: A) providing a chondrocyte having the potential for hypertrophy, and B) culturing the chondrocyte having the potential for hypertrophy on a scaffold that is biocompatible with the biological organism. The chondrocyte having the potential for hypertrophy can be cultured on a surface, or within an internal pore, of the biocompatible scaffold, preferably at 37° C. in the presence of 5-10% CO₂.

In one preferred embodiment, step A) can comprise providing the chondrocyte having the potential for hypertrophy, wherein the potential for hypertrophy is indentifed by the expression of at least one selected from, but not limited to the group consisting of type X collagen, alkaline phosphatase, osteonectin, type II collagen, cartilage proteoglycan or components thereof, hyaluronic acid, type IX collagen, type XI collagen, and chondromodulin, as a marker.

In other preferred embodiment, the step A) can comprise providing the chondrocyte having the potential for hypertrophy, wherein the potential for hypertrophy is indentifed using its hypertrophy as a marker. The hypertrophy can be observed under a microscope after preparing a pellet of the cells by centrifugation of 5×10⁵cells in HamF12 culture medium, and culturing the pellet directly.

“HamF12 growth medium” as used herein, refers to HamF12 medium containing 100 U/ml penicillin, 0.1 mg/L streptomycin, and 0.25 μg/ml amphotericin B, supplemented with 10% fetal bovine serum.

“MEM growth medium” as used herein, refers to MEM medium containing 100 U/ml penicillin, 0.1 mg/L streptomycin, and 0.25 μg/ml amphotericin B, supplemented with 15% fetal bovine serum.

In the present invention, the culture of the chondrocyte having the potential for hypertrophy is prepared using cells isolated or induced by a methods as described above. The chondrocyte having the potential for hypertrophy according to the present invention may be cultured on the surface, or if the scaffold includes the internal pore, within the pore, of a scaffold that is biocompatible with a biological organism. The cell density of the chondrocyte having the potential for hypertrophy can be adjusted, for example, to 1×10⁷ cells/ml to 1×10⁴ cells/ml. However, cell densities of less than 1×10⁴ cells/ml or more than 1×10⁷ cells/ml may also be used. Chondrocytes having the potential for hypertrophy can be proliferated in an incubator when the cell density is less than 1×10⁴ cells/ml. When the cell density of the chondrocyte having the potential for hypertrophy of the present invention is more than 1×10⁷ cells/ml, they can be used without any treatment, but optionally, they can be inoculated to a larger scaffold or diluted to an appropriate concentration in culture media. In one embodiment of the present invention, the cell density of the chondrocyte having the potential for hypertrophy can be for instance, 0.5-1×10⁶/cm³ (ml), or 1×10⁵/cm³ (ml). In another embodiment of the present invention, the cell density of a chondrocyte having the potential for hypertrophy can be 4×10⁴/cm³ (ml).

The medium used in the present invention may be any media in which the chondrocyte having the potential for hypertrophy can be proliferated. For example, such medium includes, but is not limited to: Ham's F12 (HamF12), Dulbecco's modified Eagle medium (DMEM), minimum essential medium (MEM), minimum essential medium-alpha (alpha-MEM), Eagle basal medium (BME), Fitton-Jackson modified medium (BGJb) or a combination thereof.

In another embodiment, the medium used in culturing the chondrocyte having the potential for hypertrophy in the present invention may contain any substance which enhances the proliferation, differentiation or both of cells. For example, such substances include, but are not limited to those such as at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF) and others.

In another embodiment, the chondrocyte having the potential for hypertrophy may be obtained from any region including the chondro-osseous junction of costa, epiphysial line of long bone (e. g., femoris, tibia, fibula, humerus, ulna, and radius), epiphysial line of vertebra, zone of proliferating cartilage of ossicle (e. g., hand bones, foot bones and sterna), perichondrium, bone primordium formed from cartilage of fetus, the callus region of a healing bone-fracture and the cartilaginous part of bone proliferation phase.

The scaffold that is biocompatible with a biological organism used in the present invention may be any scaffold as long as it is biocompatible, as mentioned above.

As used herein, “fixing” of cells to the scaffold refers to a condition in which the integrity of the scaffold and the cells is maintained after the cells are inoculated to the scaffold. The cells are determined to be fixed to the scaffold by observing that the integrity of the inoculated cells and the scaffold is maintained after the scaffold to which the cells inoculated is moved to another condition (for example, another medium). The another condition may be, for example, a container including the same kind of medium as that previously used during culture. When studying compatibility for transplantation, preferred conditions include, but are not limited to those into which samples are to be transplanted.

In the method according to the claimed invention, the period for culturing the chondrocyte having the potential for hypertrophy on the scaffold that is biocompatible with biological organism may be a period that is sufficient to fix the cell having the potential for hypertrophy to the scaffold. This period is preferably, but is not limited to 3 hours to 3 months. More preferably, the period is 3 hours to 3 weeks, still more preferably half a day to one week. The period for culturing the chondrocyte having the potential for hypertrophy may be less than 3 hours since the cell can adhere to the biocompatible scaffold within at least 1 hour. The period for culturing the chondrocyte having the potential for hypertrophy may be more than 3 months since the cells can be inoculated to larger scaffold, or their cell density can be readjusted if they proliferate excessively.

In another preferred embodiment, in step B), the chondrocyte having the potential for hypertrophy can be cultured in the media containing a substance for enhancing the proliferation, differentiation or both of the cell, or both.

In another preferred embodiment, in step b), the chondrocyte having the potential for hypertrophy can be cultured in media containing at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF)

(Kit)

In another aspect, the present invention provides a kit of the composite material for enhancing or inducing osteogenesis in a biological organism, comprising: A) a composite material including a chondrocyte having the potential for hypertrophy and a scaffold that is biocompatible with the biological organism, and B) a delivery means. The composite material used in the kit of the present invention can be used in any form described in the above section “Composite Material.”

(Use of the Composite Material)

In other aspect, the present invention provides uses of a composite material to produce an implant or a bone repairing material for enhancing or inducing osteogenesis in a biological organism, wherein the composite material comprises: A) a chondrocyte having the potential for hypertrophy, and B) a scaffold that is biocompatible with the biological organism. For the chondrocyte having the potential for hypertrophy and the scaffold that is biocompatible with the biological organism utilizing in the use of the composite material of the invention, any form described in the above section “Composite Material” can be used.

(Methods of Treatment)

In another aspect, the present invention provides a method of repairing a defective region of bone, which comprises implanting a composite material including a chondrocyte having the potential for hypertrophy and a scaffold that is biocompatible with the biological organism, into the defective region of the bone. The defective region of the bone may have a size that is incapable of being repaired by fixation alone. The method for implanting used in this method comprises, but is not limited to visually implanting the composite material into a defective site of bone during an operation. For the composite material used in the method for treatment according to the present invention, any form described in the above section “Composite Material” can be used. For example, the composite material of the present invention can be implanted into a biological organism together with medical device such as an implant (e. g., artificial joint), a plate, a cage, a nail, or a pin.

(Methods of Preparing a Chondrocyte having the Potential for Hypertrophy)

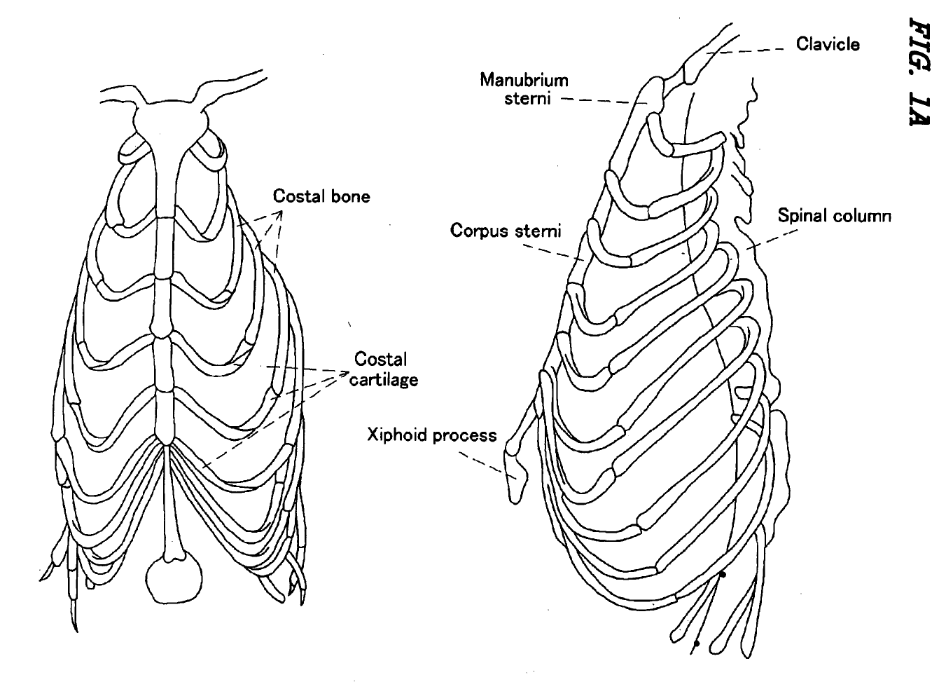

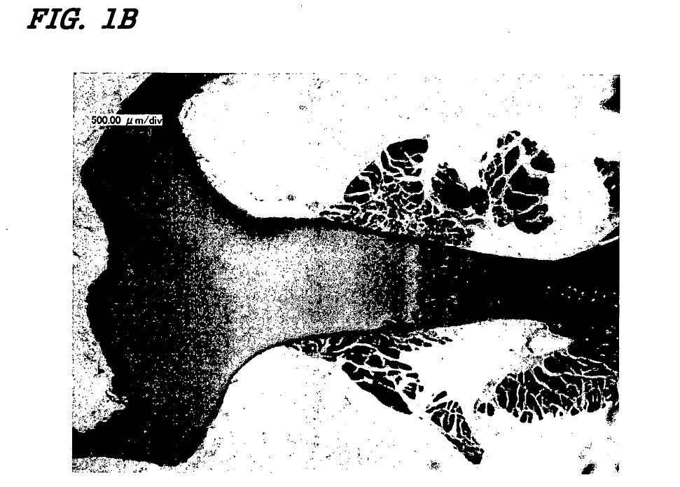

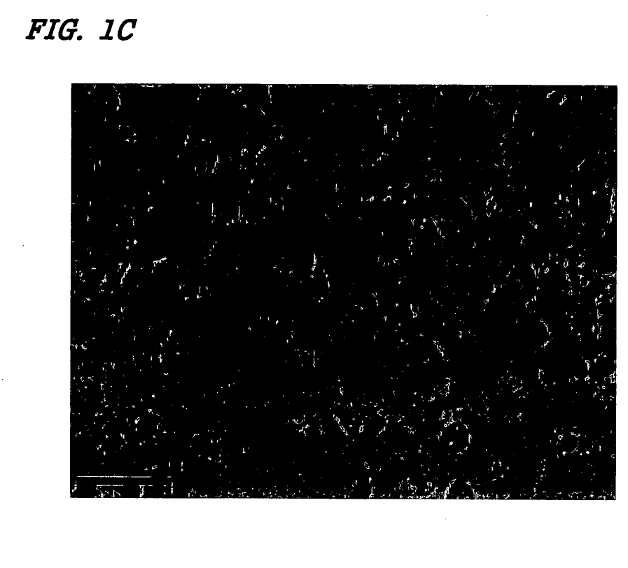

In another aspect, the present invention provides a method for preparing a chondrocyte having the potential for hypertrophy. This method comprises obtaining cells from the processus xiphoideus junction located in the inferior portion of the corpus sterni. In the present invention, “the processus xiphoideus junction located in the inferior portion of the corpus sterni” refers to the border zone between the inferior portion of the corpus sterni. (bone portion) and the processus xiphoideus (cartilage portion, which is also referred to as chondroxiphoid). While the sterni have been conventionally believed to contain growth cartilage cells, such cells have not been collected so far. I have found that “the processus xiphoideus junction located in the inferior portion of the corpus sterni” contains growth cartilage cells (FIG. 1B), and that the cells are easily obtainable therefrom. Therefore, the present invention provides a novel method for preparing growth cartilage cells. I also found that, “the processus xiphoideus junction located in the inferior portion of the corpus sterni,” is as rich as or much richer in growth cartilage cells than the costa/costal cartilage that is a conventional source of growth cartilage cells, and that the growth cartilage cells can be easily collected therefrom.

Hereinafter, the present invention will be described by way of examples. The Examples described below are provided only for illustrative purposes. Accordingly, the scope of the present invention is not limited by the above-described embodiments or the examples below, and instead is limited only by the appended claims.

### EXAMPLES

### Example 1

### Effect of Subcutaneous Implantation of a Composite Material Using Chondrocytes having the Potential for Hypertrophy Derived from Costa/Costal Cartilage and a Biocompatible Scaffold

(Preparation of Chondrocytes having the Potential for Hypertrophy from Costa/Costal Cartilage)

Male rats (Wistar) that were 4-8 weeks old were sacrificed using chloroform. The rats' chests were shaved using a razor and their whole bodies immersed in Hibitane (10-fold dilution) to be disinfected. The rats' chests were incised and the costa/costal cartilage removed aseptically. The translucent growth cartilage region was collected from the boundary region of the costa/costal cartilage. The growth cartilage was sectioned and incubated in 0.25% trypsin-EDTA/Dulbecco's phosphate buffered saline (D-PBS) at 37° C. for 1 hour, with stirring. The composition of D-PBS is KCl 0.20g/L, NaH₂PO₄ 0.20g/L, NaCl 8.00 g/L, Na₂HPO₄.7H₂O 2.16 g/L. The sections were then washed and collected by centrifugation (1000 rpm (170×g)×5 min.), followed by incubation in 0.2% Collagenase (Invitrogen)/D-PBS at 37° C. for 2.5 hours, with stirring. After collection by centrifugation (1000 rpm (170×g)×5 min.), the cells were incubated in HamF12 growth medium (i. e. HamF12 containing 100 U/ml penicillin, 0.1 mg/L streptomycin, and 0.25 μg/ml amphotericin B, supplemented with 10% fetal bovine serum) with 0.2% Dispase (Invitrogen) in a stirring flask overnight at 37° C. with stirring. The following day, the resulting cell suspension was filtered and the cells washed and collected by centrifugation (1000 rpm (170×g)×5 min.). The cells were stained with trypan blue and counted under a microscope.

The cells were evaluated, cells not stained were considered to be living cells, and those stained blue were considered to be dead cells.

(Identification of Chondrocytes having the Potential for Hypertrophy)

Since the cells obtained in Example 1 are impaired by the enzymes used in cell separation (e. g. trypsin, collagenase, and dispase), they are cultured to recover. Chondrocytes having the potential for hypertrophy are identified by using their expression of chondrocyte markers and their morphological hypertrophy under a microscope.

(Expression of Specific Markers for Chondrocytes having the Potential for Hypertrophy)

A cell suspension prepared using a method as described above is treated with sodium dodecyl sulfate (SDS). The SDS-treated solution is subjected to SDS polyacrylamide gel electrophoresis. The gel is blotted onto a transfer membrane (Western blotting), reacted with a primary antibody to chondrocyte markers, and detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methylcoumarin-3-acetate (AMCA) or rhodamine.

Cell cultures prepared using a method as described above are fixed with 10% neutral formalin buffer, reacted with a primary antibody to chondrocyte markers, and detected with a secondary antibody labeled with an enzyme such as peroxidase, alkaline phosphatase or glucosidase, or fluorescent tag such as FITC, PE, Texas Red, AMCA or rhodamine.

(Histological Assessment of the Potential for Hypertrophy in Chondrocytes)

5×105 cells in HamF12 growth medium were centrifuged to prepare a pellet of cells. The pellet was cultured for a pre-determined period, fixed with 10% neutral buffered formalin, and embedded in paraffin. The sample was sectioned and stained with hematoxylin and eosin (HE stain). Cell size before and after culture was compared under a microscope. When significant proliferation was observed, the cells were determined to have the potential for hypertrophy (FIG. 1C).

(Analysis Using Marker Genes)

Chondrocytes having the potential for hypertrophy obtained in the present Example are analyzed for the amount of mRNA transcripts of type X collagen, alkaline phosphatase, type II collagen, or cartilage proteoglycan, which are markers for chondrocytes having the potential for hypertrophy. mRNA species are detected using real-time PCR as below.

(Real-Time PCR)

Chondrocytes having the potential for hypertrophy obtained in the present Example are used as a sample, inoculated to hydroxyapatite in HamF12 growth medium (1/106 cells/ml), and cultured in a 5% CO₂ incubator at 37° C. for one week. Chondrocytes (1/10⁶ cells/ml) are used as a control. Total RNA is extracted from the sample.

The samples are put into milling vessel with liquid-nitrogen and milled with a milling machine. Then, the samples are transferred into a 2.0 ml tube, 1 ml of ISOGEN (Wako Pure Chemical Industries Ltd.) is added to the samples, the samples mixed with a vortex mixer, and ground with a Polytron until homogenized. After the samples are incubated at RT for 5 minutes, they are vortexed vigorously with 0.2 ml chloroform. After further incubation at 4° C. for 5 minutes, the samples are centrifuged at 12,000×g, 4° C. for 15 minutes. The aqueous phase is collected from the tube, and vortexed with 0.6 ml isopropanol. The solution is incubated at RT for 10 minutes, then stored at −30° C. overnight. The following day, the solution are centrifuged at 12,000×g, 4° C. for 15 minutes, then the supernatant are removed, dried, and washed with 1 ml of 75% ethanol to yield total RNA.

cDNA is synthesized from the total RNA using a High-Capacity cDNA Archive Kit (Applied Biosystems). Type X collagen, alkaline phosphatase, type II collagen and cartilage proteoglycan are purchased from Applied Biosystems. Then, using cDNA as template, the expression of type X collagen, alkaline phosphatase, type II collagen and cartilage proteoglycan are detected using the Taqman assay (Taqman® Gene Expression Assays (Applied Biosystems)).

Real-time PCR solution (25 μL of 2×TaqMan Universal PCR Master Mix, 2.5 μL of 20×Taqman® Gene Expression Assay Mix, 21.5 μL of RNase-free water, and 1 μL of cDNA template) is prepared and dispensed into a 96 well-PCR plate. Then, a PCR is run for 40 cycles of 2 minutes at 50° C., 15 seconds at 95° C., and 1 minute at 60° C., using PCR Master Mix (Applied Biosystems). Data are detected with a Real-time PCR cycler (ABI PRISM 7900 HT). After PCR, setting the threshold and calculating the threshold cycle was performed using analytical software provided with the instrument (PRISM 7900 HT).

(Results)

It is found that, in chondrocytes having the potential for hypertrophy, type II collagen and cartilage proteoglycan (i. e. chondrocyte markers) are expressed, and that the expression of type X collagen and alkaline phosphatase (i. e. markers for chondrocyte having the potential for hypertrophy) is significantly higher in chondrocytes having the potential for hypertrophy than in the chondrocyte control.

(Identification of Cells having the Potential for Hypertrophy)

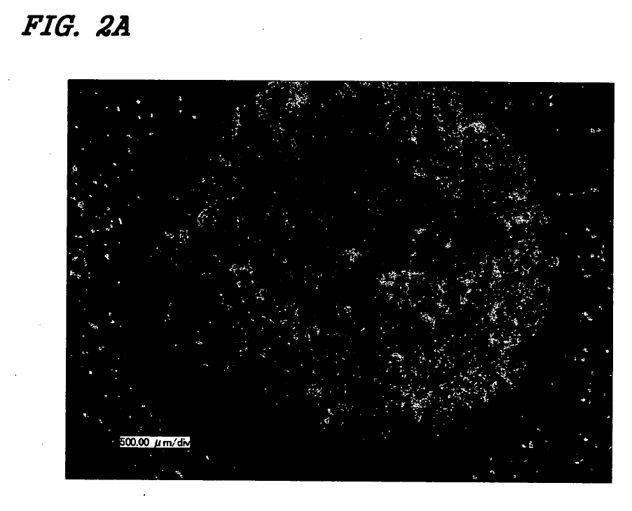

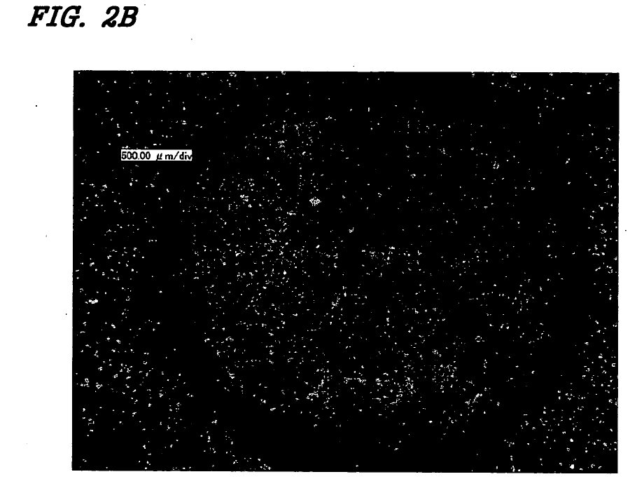

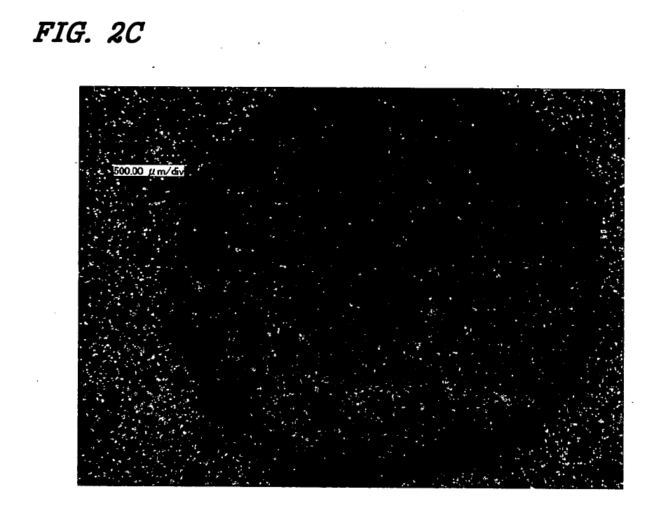

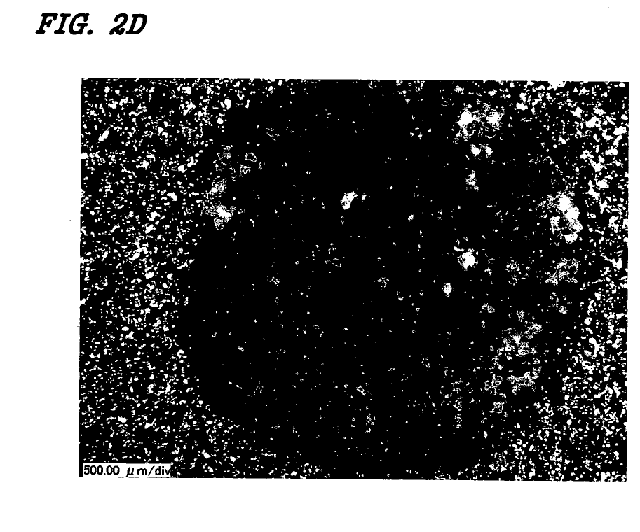

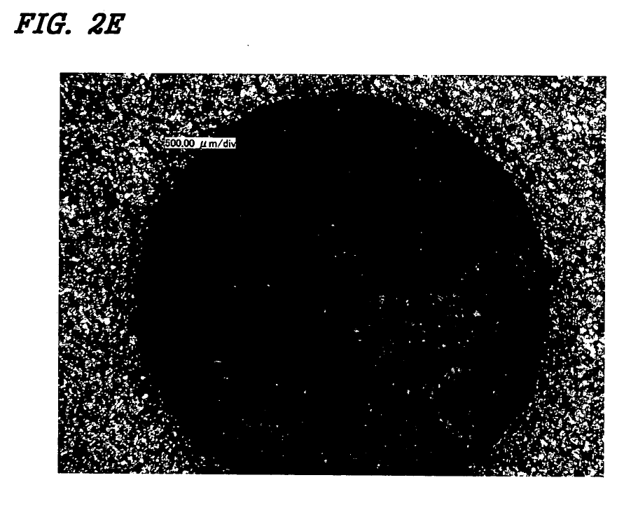

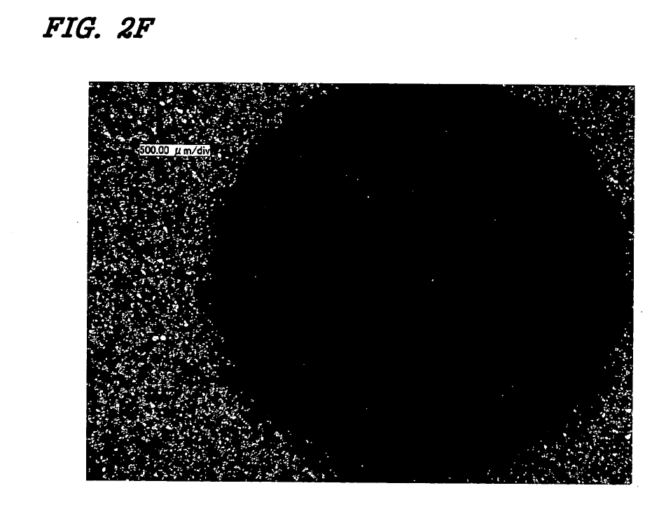

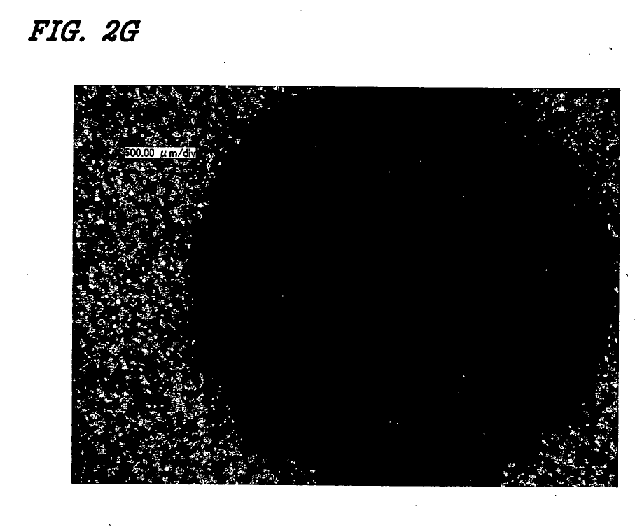

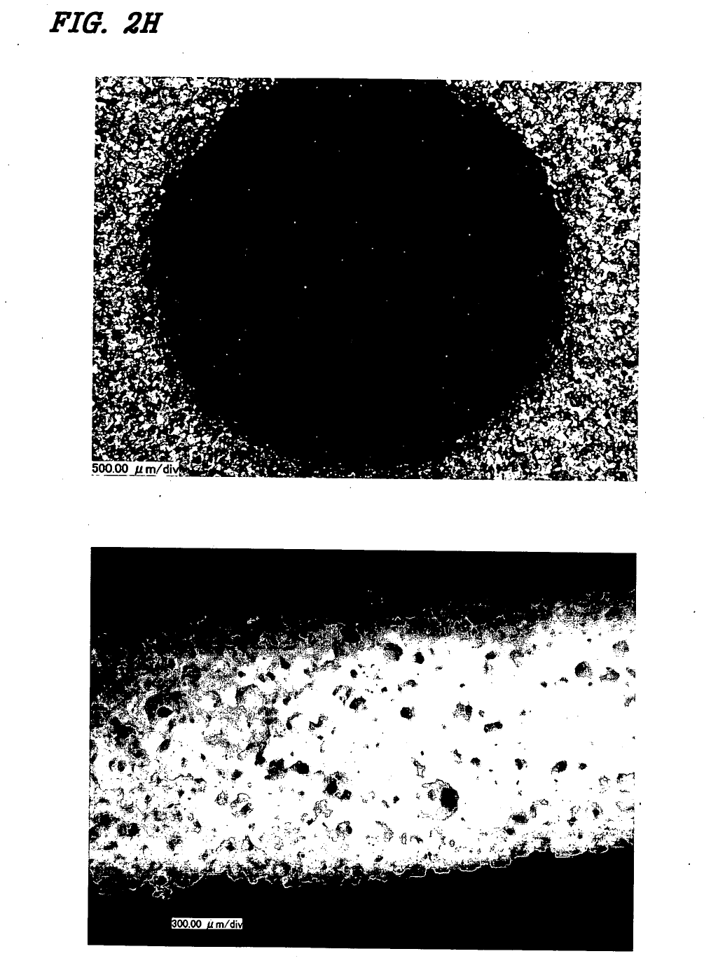

To determine whether chondrocytes having the potential for hypertrophy existed in cell suspensions in which chondrocytes having the potential for hypertrophy were diluted, the following experiment was performed. Chondrocytes having the potential for hypertrophy (1×10⁶ cells/ml) were inoculated to seven blocks of discoid hydroxy apatite having 85% porosity (5 mm in diameter)/24-well plate (1.43×10⁵ cells/discoid hydroxyapatite), and cultured in HamF12 growth medium, in a 5% CO₂ incubator at 37° C. for 3 hours, 1 day, 3 days, or one week. These samples (hydroxyapatite inoculated with cells) were then stained with alkaline phosphatase, fixed with 10% neutral buffered formalin, and stained with toluidine blue. For alkaline phosphatase staining, the samples were fixed by immersion in 60% acetone/citric acid buffer for 30 second, rinsed in water, and incubated with alkaline phosphatase stain solution (2ml of 0.25% naphthol AS-MX alkaline phosphate (Sigma-Aldrich) +48 ml 0.025% First Violet B salt solution (Sigma-Ardrich)) at RT in the dark for 30 minutes. For toluidine blue staining, the samples were incubated with toluidine blue stain solution (0.05% toluidineblue solution, pH 7.0, Wako Pure Chemical Industries Ltd.), at RT for 3-10 min. For all culture periods, the samples displayed red spotted staining with alkaline phosphate (see, FIGS. 2A-D). With toluidine blue, the same site of the samples displayed blue spotted staining for all culture periods, showing the existence of cells having the desired properties (see, FIGS. 2E-H). Thus, it was found that cells existing on hydroxyapatite have alkaline phosphatase activity.

(Results)

The cells obtained in the present Example expressed a chondrocyte marker, and were determined to be morphologically hypertrophic. This shows that the cells obtained in the present Example were chondrocytes having the potential for hypertrophy. These cells were used in the following experiments.

(Producing a Composite Material Using Chondrocytes having the Potential for Hypertrophy Obtained from Costa/Costal Cartilage and a Biocompatible Scaffold)

HamF12 growth medium was added to chondrocytes having the potential for hypertrophy obtained in the present Example, to a final cell density of 1×10⁶ cells/ml. The cell suspension was inoculated evenly to gelatin, collagen and hydroxyapatite, respectively, and cultured in a 5% CO₂ incubator at 37° C. for one week.

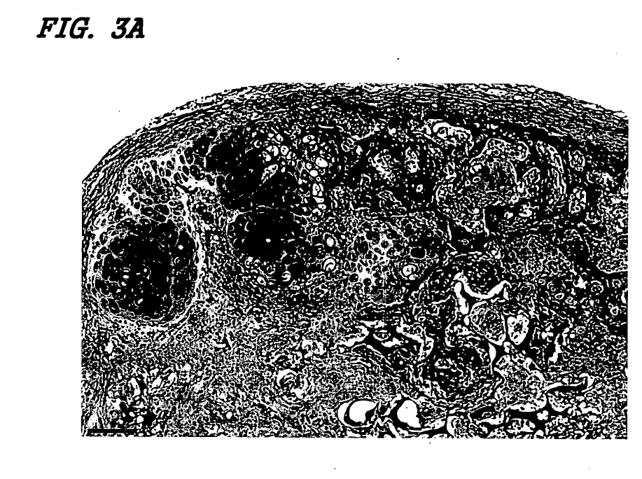

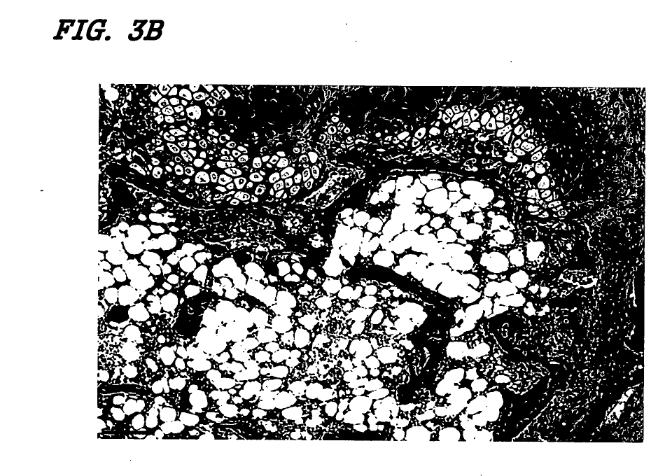

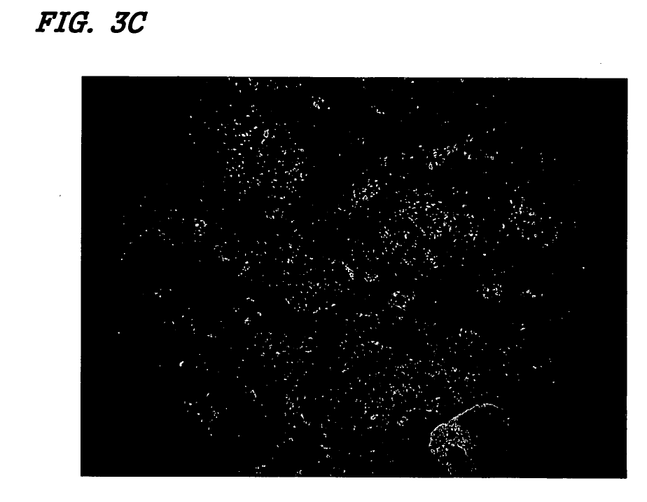

These cultures were subcutaneously implanted into rats. Four weeks after implantation, the rats were sacrificed and the implanted region removed, fixed with 10% neutral buffered formalin, and embedded in paraffin. The sample was sectioned. and stained with HE to evaluate the condition of the implanted region. Osteogenesis was observed in all of the composite materials using biocompatible scaffolds made of gelatin (FIG. 3A), collagen (FIG. 3B) and hydroxyapatite (FIG. 3C), respectively.

### Comparative Example 1A

### Effect of Subcutaneous Implantation of a Pellet of Chondrocytes having the Potential for Hypertrophy Derived from Costa/Costal Cartilage

(Preparation of Pellet of Chondrocytes having the Potential for Hypertrophy Derived from Costa/Costal Cartilage)

Chondrocytes having the potential for hypertrophy were collected from costa/costal cartilage by a method as described in Example 1. HamF12 growth medium was added to these cells (5×10⁵ cells) to a final cell density of 5×10⁵ cells/0.5 ml. The cell suspension was centrifuged at (1000 rpm (170×g)×5 min.) to prepare a pellet of chondrocytes having the potential for hypertrophy.

The pellet of chondrocytes having the potential for hypertrophy was subcutaneously implanted into rats. Four weeks after implantation, the rats were sacrificed and the implanted region removed, fixed with 10% neutral buffered formalin, and embedded in paraffin. The sample was sectioned and stained with HE to evaluate the condition of the implanted region. Slight osteogenesis was observed when the pellet of chondrocytes having the potential for hypertrophy was implanted, however, the area was significantly smaller compared to when composite material using chondrocytes having the potential for hypertrophy from costa/costal cartilage and a biocompatible scaffold, was implanted (see, Example 1).

### Comparative Example 1B

### Effect of Subcutaneous Implantation of Chondrocytes having the Potential for Hypertrophy Derived from Costa/Costal Cartilage Alone

Chondrocytes having the potential for hypertrophy obtained by a method as described in Example 1 were subcutaneously implanted alone into rats. Osteogenesis was not observed when the chondrocytes having the potential for hypertrophy were implanted alone.

### Comparative Example 1C

### Effect of Subcutaneous Implantation of Hydroxyapatite Alone

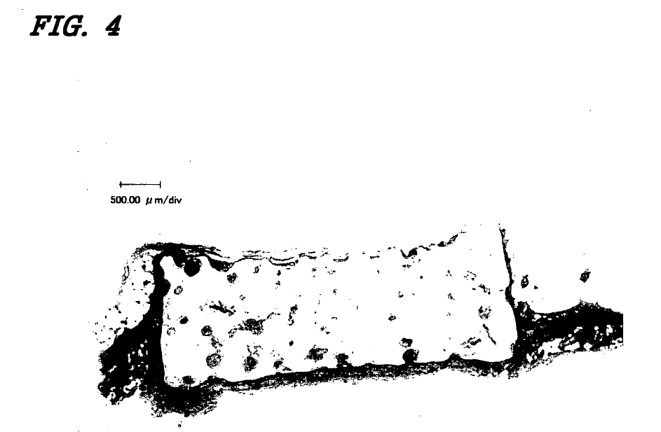

Hydroxyapatite (scaffold) was subcutaneously implanted into rats, alone, using a method as described in Example 1. Osteogenesis was not observed when hydroxyapatite was implanted alone (FIG. 4).

### Summary of Example 1 and Comparative Examples 1A-1C

Osteogenesis was more prominent after the subcutaneous implantation of composite material using chondrocytes having the potential for hypertrophy and a biocompatible scaffold into rats, compared with the pelleted chondrocytes having the potential for hypertrophy alone. In contrast, when the chondrocytes having the potential for hypertrophy or hydroxyapatite were implanted alone, osteogenesis was not observed. These results suggest that the composite material of the present invention can be used to treat bone deficits which are too large to treat with conventional composite materials.

### Example 2

### Effect of Subcutaneous Implantation of a Composite Material Using Chondrocytes having the Potential for Hypertrophy Derived from Sterna and a Biocompatible Scaffold

(Preparation of Chondrocytes having the Potential for Hypertrophy from Sterna)

Male rats (Wistar) that were 4-8 weeks old were sacrificed using chloroform. The rats' chests were shaved using a razor and their whole bodies immersed in Hibitane (10-fold dilution) to be disinfected. The rats' chests were incised and the processus xiphoideus junction located in the inferior portion of the corpus sterni and other regions were removed from sterna aseptically. The translucent growth cartilage region was collected from the processus xiphoideus junction located in the inferior portion of the corpus sterni. These samples were sectioned and incubated in 0.25% trypsin-EDTA/D-PBS at 37° C., with stirring, for 1 hour. The sections were then washed and collected by centrifugation (1000 rpm (170×g)×5 min.) followed by incubation in 0.2% Collagenase/D-PBS at 37° C. for 2.5 hours, with stirring. After collection by centrifugation (1000 rpm (170×g)×5 min.), the cells were incubated with 0.2% Dispase/HamF12 growth medium in a stirring flask overnight at 37° C. with stirring. Optionally, the overnight treatment with 0.2% Dispase was omitted. On the following day, the cell suspension was filtered and washed and collected by centrifugation (1000 rpm (170×g)×5 min.). The cells were stained with trypan blue and counted under a microscope.

The cells were evaluated, cells not stained were considered to be living cells, and those stained blue were considered to be dead cells.

(Identification of Chondrocytes having the Potential for Hypertrophy)

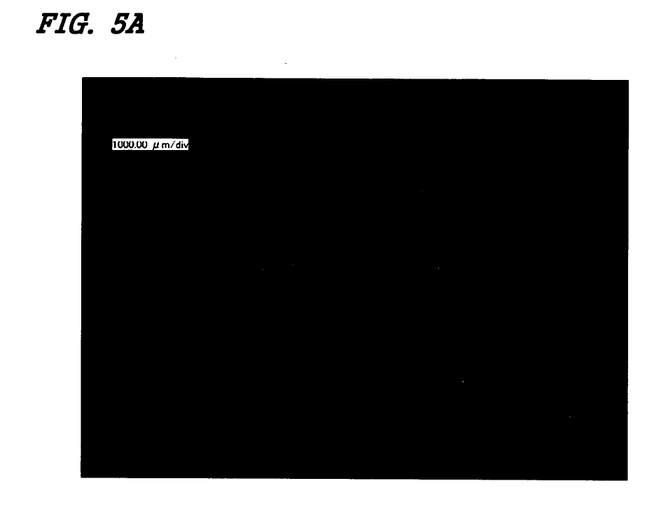

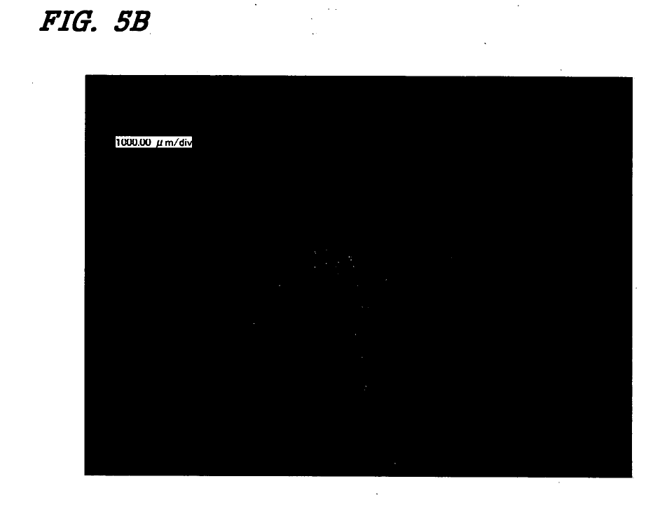

Using a method as described in Example 1, the collected cells were identified as being chondrocytes having the potential for hypertrophy (FIG. 5A). The chondrocytes having the potential for hypertrophy were not observed in regions other than the growth cartilage region of the sterna (FIG. 5B).

(Producing a Composite Material Using Chondrocytes having the Potential for Hypertrophy Obtained from Sterna and a Biocompatible Scaffold)

Using the chondrocytes having the potential for hypertrophy obtained in the present Example, a composite material is prepared by a method as described in Example 1, and subcutaneously implanted into rats. Four weeks after implantation, the rats are sacrificed and the implanted region removed, fixed with 10% neutral buffered formalin, and embedded in paraffin. The sample is sectioned and stained with HE to evaluate the condition of the implanted region. Osteogenesis is observed in all of the composite materials using the biocompatible scaffolds made of gelatin, collagen and hydroxyapatite, respectively.

### (Comparative Example 2A

### Effect of Subcutaneous Implantation of the Pellet of Chondrocytes having the Potential for Hypertrophy Derived from Sterna)

(Preparation of Pellet of Chondrocytes having the Potential for Hypertrophy from Sterna)

Chondrocytes having the potential for hypertrophy are collected from sterna by a method as described in Example 2. HamF12 growth medium is added to these cells (5×10⁵ cells) to dilute to a cell density of 5×10⁵ cells/0.5 ml. The cell suspension is centrifuged (1000 rpm (170×g)×5 min) to prepare a pellet of chondrocytes having the potential for hypertrophy.

The pellet of chondrocytes having the potential for hypertrophy is subcutaneously implanted into rats. Four weeks after implantation, the rats are sacrificed and the implanted region removed, fixed with 10% neutral buffered formalin, and embedded in paraffin. The sample is sectioned and stained with HE to evaluate the condition of the implanted region. Slight osteogenesis is observed when the pellet of the chondrocytes having the potential for hypertrophy from sterna is implanted, however, the area is significantly smaller compared to the composite material using chondrocytes having the potential for hypertrophy derived from sterna and a biocompatible scaffold, is implanted (see, Example 2).

### Comparative Example 2B

### Effect of Subcutaneous Implantation of Chondrocytes having the Potential for Hypertrophy Derived from Sterna Alone)

Chondrocytes having the potential for hypertrophy obtained by a method as described in Example 2, are implanted alone subcutaneously into rats. Osteogenesis is not observed when the chondrocytes having the potential for hypertrophy were implanted alone.

### Summary of Example 2, and Comparative Examples 2A, 2B and 1C

Osteogenesis is more prominent after the subcutaneous implantation of composite materials using chondrocytes having the potential for hypertrophy and a biocompatible scaffold into rats, compared with the pelleted chondrocytes having the potential for hypertrophy, alone. On the other hand, when chondrocytes having the potential for hypertrophy, or hydroxyapatite is implanted alone, osteogenesis is not observed (see, Comparative Example 1C). These results suggest that the composite material of the present invention can be used to treat bone deficits which are too large to treat with prior art methods.

### Comparative Example 3A

### Effect of Subcutaneous Implantation of a Composite Material Using Chondrocytes without the Potential for Hypertrophy Derived from Auricular Cartilage and a biocompatible Scaffold

(Preparation of Chondrocytes without the Potential for Hypertrophy from Auricular Cartilage)

Male rats (Wistar) of 4-8 weeks old are sacrificed using chloroform. The rats' whole bodies are immersed in Hibitane (10-fold dilution) to be disinfected. The rats are incised around auricular cartilage, the skin ablated and the auricular cartilage removed, aseptically. The auricular cartilage is sectioned and incubated in 0.25% trypsin-EDTA/D-PBS at 37° C. for 1 hour, with stirring. The sections are then washed and collected by centrifugation (1000 rpm (170×g)×5 min.) followed by incubation with 0.2% Collagenase/D-PBS at 37° C. for2.5 hours, with stirring. After collection by centrifugation (1000 rpm (170×g)×5 min.), the cells are incubated with 0.2% Dispase/HamF12 growth medium in a stirring flask overnight at 37° C. with stirring. Optionally, the overnight treatment with 0.2% Dispase is omitted. On the following day, the cell suspension is filtered and the cells washed and collected by centrifugation (1000 rpm (170×g)×5 min.). The cells are stained with trypan blue and counted under a microscope.

The cells are evaluated, cells not stained are considered to be living cells, and those stained in blue are considered to be dead cells.

(Identification of Chondrocytes without the Potential for Hypertrophy Derived from Auricular Cartilage)

Using a method as described in Example 1, it is determined whether chondrocytes having the potential for hypertrophy are found in cell suspensions obtained by diluting chondrocytes without the potential for hypertrophy derived from auricular cartilage, or not. The sample is not stained with alkaline phosphatase. With toluidine stain, the sample displays blue spotted staining, showing the existence of cells, which indicated that the cells existing on the hydroxyapatite do not have alkaline phosphatase activity. Thus, the cell suspension used in the present Comparative Example contained only chondrocytes without the potential for hypertrophy.

By detecting the localization or expression of chondrocyte markers using a method as described in Example 1, and examining the cells morphologically, it is determined that the cells obtained are chondrocytes without the potential for hypertrophy.

(Producing a Composite Material Using Chondrocytes without the Potential for Hypertrophy Obtained from Auricular Cartilage and a Biocompatible Scaffold

Using the chondrocytes without the potential for hypertrophy obtained in the present Comparative Example, a composite material is prepared by a method as described in Example 1, and subcutaneously implanted into rats. Four weeks after implantation, the rats are sacrificed and the implanted region removed, fixed with 10% neutral buffered formalin, and embedded in paraffin. The sample is sectioned and stained with HE to evaluate the condition of the implanted region. Osteogenesis is not observed in any of the composite materials using the biocompatible scaffolds made of gelatin, collagen and hydroxyapatite, respectively.

### Comparative Example 3B

### Effect of Subcutaneous Implantation of a Composite Material Using Chondrocytes without the Potential for Hypertrophy Derived from Articular Cartilage and a Biocompatible Scaffold

(Preparation of Chondrocytes without the Potential for Hypertrophy from Articular Cartilage

Male rats (Wistar) of 4-8 weeks old were sacrificed using chloroform. The rats were shaved around their knee joint region using a razor and their whole bodies immersed in Hibitane (10-fold dilution) to be disinfected. The rats were incised at their knee joint region and articular cartilage was removed aseptically. The articular cartilage was sectioned and stirred in 0.25% trypsin-EDTA/D-PBS at 37° C. for 1 hour. The sections were then washed and collected by centrifugation (1000 rpm (170×g)×5 min.) followed by stirring with 0.2% Collagenase/D-PBS at 37° C. for 2.5 hours. After collecting with centrifugation (1000 rpm (170×g)×5 min.), the cells were stirred with 0.2% Dispase/HamF12 growth medium in stirring flask overnight at 37° C. Optionally, the overnight treatment with 0.2% Dispase was omitted. On the following day, the cell suspension was filtered and the cells washed and collected by centrifugation (1000 rpm (170×g)×5 min.). The cells were stained with trypan blue and counted under a microscope.

The cells were evaluated, cells not stained were considered to be living cells, and those stained in blue are considered to be dead cells.

(Identification of Chondrocytes without the Potential for Hypertrophy Derived from Articular Cartilage)

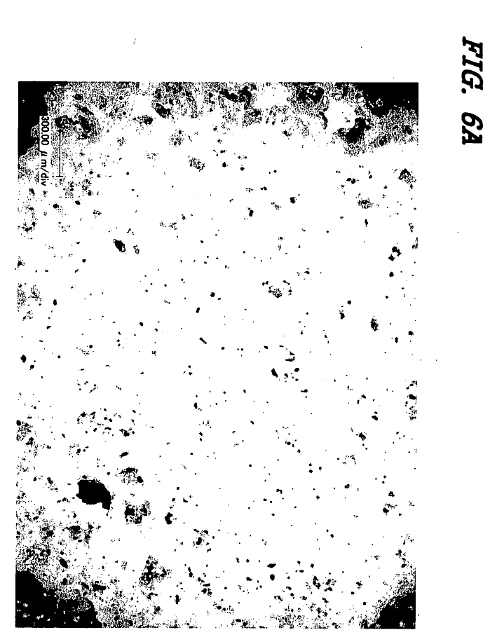

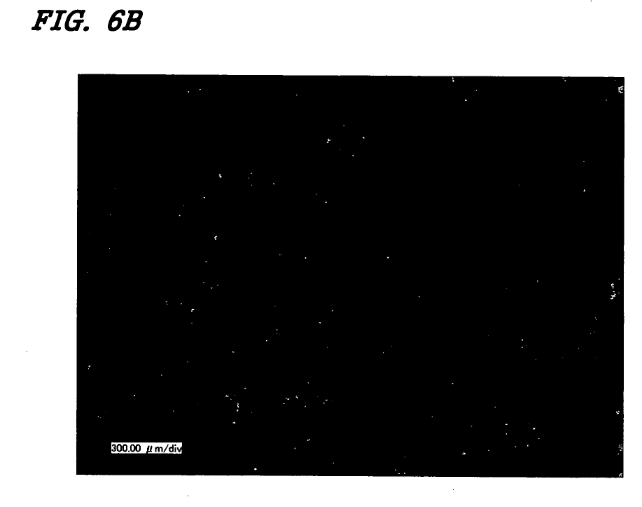

Using a method as described in Example 1, it was determined whether chondrocytes having the potential for hypertrophy were found in cell suspensions obtained by diluting chondrocytes without the potential for hypertrophy derived from articular cartilage, or not. Chondrocytes without the potential for hypertrophy (1×10⁶ cells/ml) were inoculated to seven blocks of discoid hydroxyapatite having 85% porosity (5 mm in diameter)/24-well plate (1.43×10⁵ cells/discoid hydroxyapatite), and cultured in HamF12 growth medium, in 5% CO₂ incubator at 37° C. for one week. These samples did not stain with alkaline phosphatase. With toluidine blue, the samples displayed blue spotted staining, showing the existence of cells (see, FIG. 6A). The cells existing-on the hydroxyapatite were not found to have alkaline phosphate activity (see, FIG. 6B). Therefore, it is indicated that the cell suspension used in the present Comparative Examples contained chondrocytes without the potential for hypertrophy.

By detecting the localization or expression of chondrocyte markers using a method as described in Example 1, and examining the cells morphologically, it is determined that the cells obtained are chondrocytes without the potential for hypertrophy.

(Producing a Composite Material Using Chondrocytes without the Potential for Hypertrophy Obtained from Articular Cartilage and a Biocompatible Scaffold)

Using the chondrocytes without the potential for hypertrophy obtained in the present Comparative Example, a composite material is prepared by a method as described in Example 1, and subcutaneously implanted into rats. Four weeks after implantation, the rats are sacrificed and the implanted region removed, fixed with 10% neutral buffered formalin, and embedded in paraffin. The sample is sectioned and stained with HE to evaluate the condition of the implanted region. Osteogenesis is not observed on any of the composite materials using the biocompatible scaffolds made of gelatin, collagen and hydroxyapatite, respectively.

### Comparative Example 3C

### Effect of Subcutaneous Implantation of a Composite Material Using Resting Cartilage Cells without the Potential for Hypertrophy Derived from Costal Cartilage and a Biocompatible Scaffold

(Preparation of Resting Cartilage Cells without the Potential for Hypertrophy from Costal Cartilage)

Male rats (Wistar) that were 4-8 weeks old were sacrificed using chloroform. The rat's chests were shaved using a razor and their whole bodies immersed in Hibitane (10-fold dilution) to be disinfected. The rats' chests were incised and the costal cartilage removed aseptically. The region of opaque resting cartilage was collected from the costal cartilage. The resting cartilage was sectioned and incubated in 0.25% trypsin-EDTA/D-PBS at 37° C., with stirring, for 1 hour. The sections were then washed and collected by centrifugation (1000 rpm (170×g)×3 min.) followed by incubation in 0.2% Collagenase (Invitrogen)/D-PBS at 37° C. for 2.5 hours, with stirring. After collection by centrifugation (1000 rpm (170×g)×3 min.), the cells were incubated with 0.2% Dispase (Invitrogen)/HamF12 growth medium in a stirring flask overnight at 37° C. with stirring. Optionally, the overnight treatment with 0.2% Dispase was omitted. The following day, the resulting cell suspension was filtered and the cells washed and collected by centrifugation (1000 rpm (170×g)×3 min.). The cells were stained with trypan blue and counted under a microscope.

The cells were evaluated, cells not stained were considered to be living cells, and those stained blue were considered to be dead cells.

(Identification of Resting Cartilage Cells without the Potential for Hypertrophy Derived from Costal Cartilage)

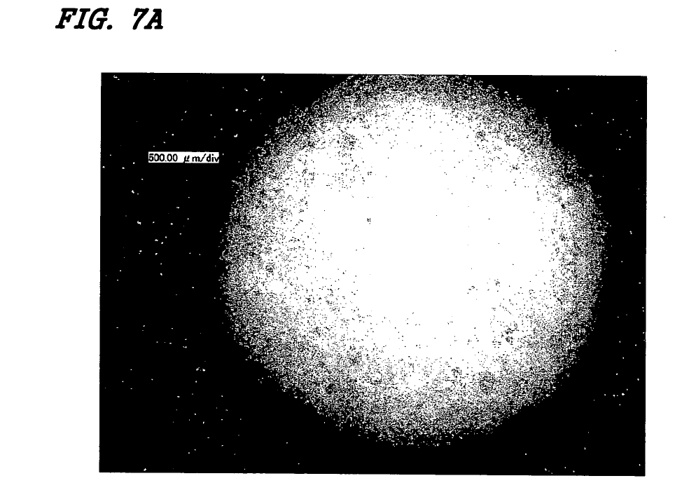

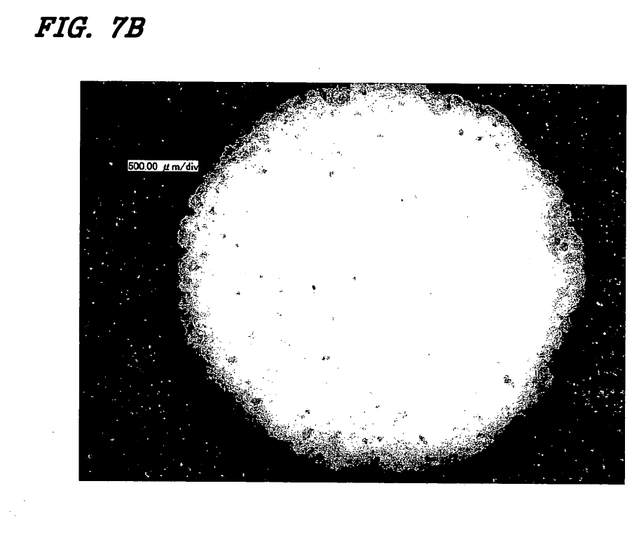

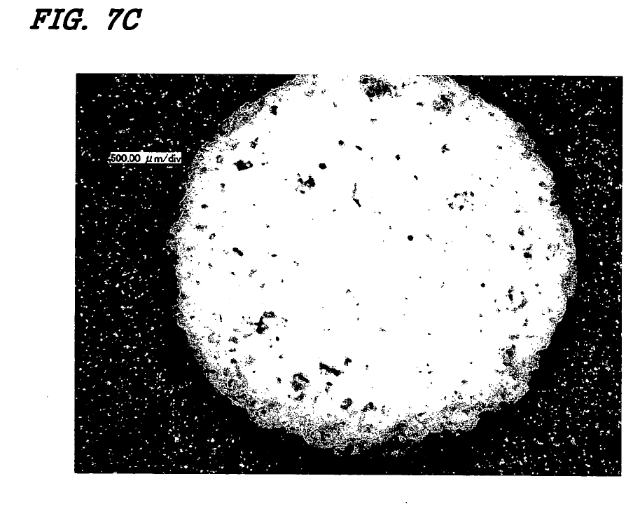

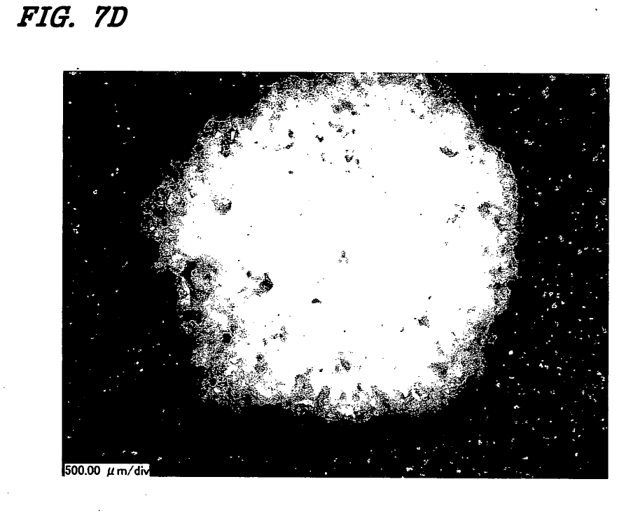

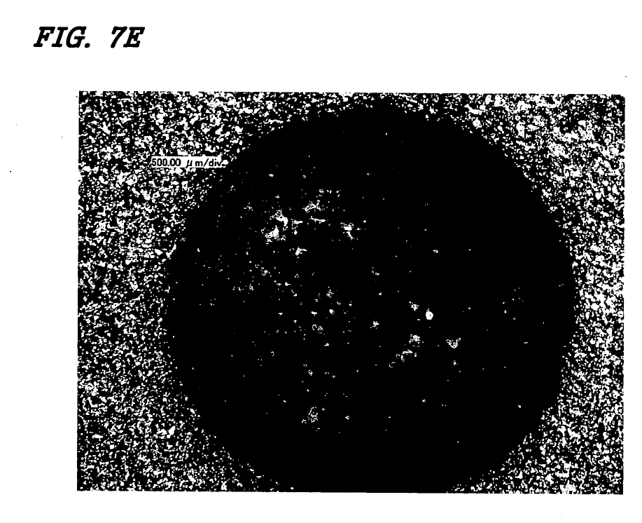

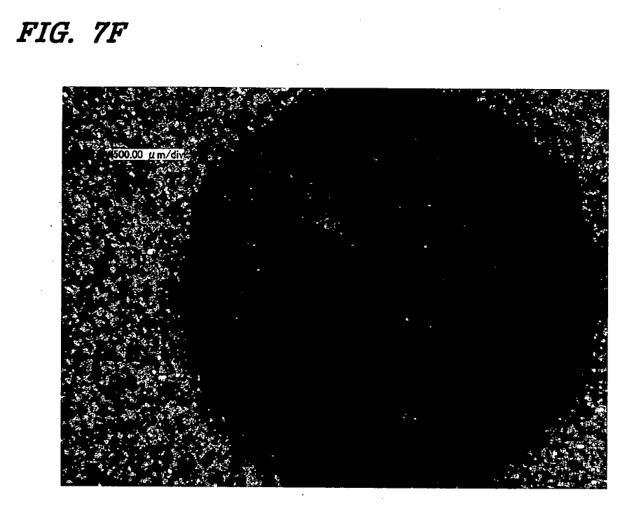

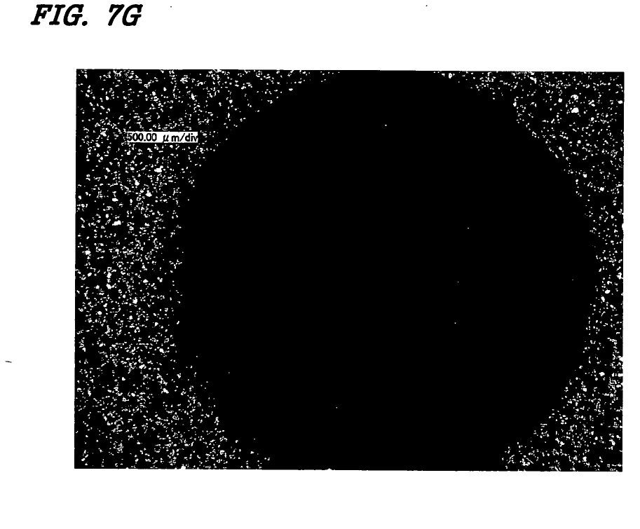

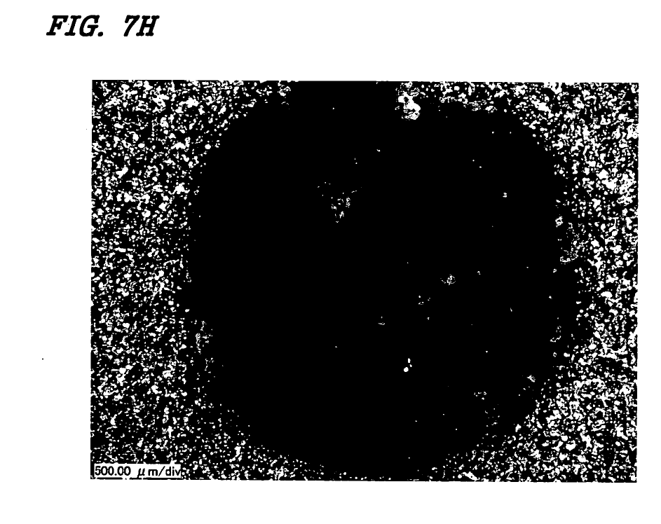

Using a method as described in Example 1, it was determined whether chondrocytes having the potential for hypertrophy were found in cell suspensions obtained by diluting resting cartilage cells without the potential for hypertrophy derived from costal cartilage, or not. Resting cartilage cells (1×10⁶ cells/ml) were inoculated to discoid blocks of hydroxyapatite having 85% porosity (5 mm in diameter)/24-well plate (1.43×10⁵cells/discoidhydroxyapatite), and cultured in HamF12 growth medium, in a 5% CO₂ incubator at 37° C. for 3 hours, 1 day, 3 days, or one week. None of the samples stained with alkaline phosphatase. With toluidine blue, the samples displayed blue spotted staining, showing the existence of cells (see, FIGS. 7A-7D). Thus, it was concluded that the cells existing on the hydroxyapatite didn't have alkaline phosphate activity (see, FIGS. 7E-7H), indicating that the cell suspension used in the present Comparative Example contained chondrocytes without the potential for hypertrophy.

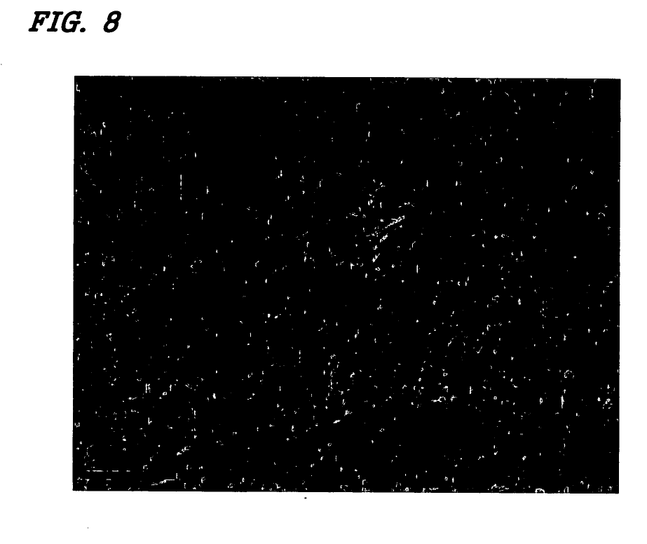

By detecting the localization or expression of chondrocyte markers using a method as described in Example 1, and examining the cells morphologically, it was determined that the cells obtained were chondrocytes without the potential for hypertrophy (FIG. 8).

(Producing a Composite Material Using Resting Cartilage Cells without the Potential for Hypertrophy Derived from Costal Cartilage and a Biocompatible Scaffold)

Using the resting cartilage cells without the potential for hypertrophy obtained in the present Comparative Example, a composite material is prepared by a method as described in Example 1, and subcutaneously implanted into rats. Four weeks after implantation, the rats are sacrificed and the implanted region removed, fixed with 10% neutral buffered formalin, and embedded in paraffin. The sample is sectioned and stained with HE to evaluate the condition of the implanted region. Osteogenesis is not observed in any of the composite materials using the biocompatible scaffolds made of gelatin, collagen and hydroxyapatite, respectively.

### Comparative Example 4

### Effect of Subcutaneous Implantation of a Composite Material Using Osteoblasts and a Biocompatible Scaffold

(Preparation of Osteoblasts)

Newborn male rats (Wistar) are sacrificed using chloroform. The rats' whole bodies are immersed in Hibitane (10-fold dilution) to be disinfected. The rats' heads are incised and the skulls removed aseptically. The skull is sectioned and incubated in 0.2% Collagenase/D-PBS at 37° C. for 1.5 hours with stirring. The cell suspension is filtered and the cells washed and isolated by centrifugation (1000 rpm (1000 rpm (170×g)×5 min.). The cells are stained with trypan blue and counted under a microscope.

The cells are evaluated, cells not stained are considered to be living cells, and those stained blue are considered to be dead cells.

(Identification of Osteoblasts)

Osteoblasts are identified using a method as described in Example 1, except for using an osteoblast marker as a marker and MEM growth medium as a medium.

(Producing a Composite Material Using Osteoblasts and a Biocompatible Scaffold)

Using osteoblasts obtained in the present Comparative Example, a composite material is prepared by a method as described in Example 1, and implanted subcutaneously into rats. Four weeks after implantation, the rats are sacrificed and the implanted region removed, fixed with 10% neutral buffered formalin, and embedded in paraffin. The sample is sectioned and stained with HE to evaluate the condition of the implanted region. Slight osteogenesis is observed in all of the composite materials using osteoblasts and the biocompatible scaffolds made of gelatin, collagen and hydroxyapatite, respectively. This osteogenesis is compared to that induced by implantation of composite materials using chondrocytes having the potential for hypertrophy derived from costa/costal cartilage and a biocompatible scaffold (see Example 1) and from sterna and a biocompatible scaffold (see Example 2). These comparisons show that composite material using chondrocytes having the potential for hypertrophy and a biocompatible scaffold have superior osteogenetic ability to composite materials using osteoblasts.

### Comparative Example 5

### Effect of Subcutaneous Implantation of a Composite Material Using Mesenchymal Stem Cells or Osteoblasts Derived from Mesenchymal Stem Cells and a Biocompatible Scaffold

(Preparation of Mesenchymal Stem Cells)

Rats (Wistar) of 4-8 weeks old are used as subject. The rat's femur is aseptically removed, the ends removed, and is sacrificed and their bone marrow washed with MEM growth medium. The washed bone marrow cells are inoculated into a 75 cm³ culture flask (T-75). After incubation in 5% CO₂, at 37° C. for one week to ten days, cells adhering to the flask are used in the following experiments as mesenchymal stem cells derived from bone marrow.

(Production of a Composite Material Using Mesenchymal Stem Cells or Osteoblasts Derived from Mesenchymal Stem Cells and a Biocompatible Scaffold)

A composite material is prepared using a method as described in Example 1, except for using mesenchymal stem cells obtained in the present Comparative Example and MEM growth medium. The composite material is further incubated in MEM differentiation medium, in 5% CO₂ at 37° C. for 2 weeks, to differentiate the mesenchymal stem cells on or within the biocompatible scaffold into osteoblasts. A composite material using the differentiated osteoblasts and hydroxyapatite is subcutaneously implanted into rats. A composite material using a biocompatible scaffold and mesenchymal stem cells that do not undergo the 2 weeks differentiation procedure, is also used for implantation. Four weeks after implantation, the rats are sacrificed and the implanted region removed, fixed with 10% neutral buffered formalin, and embedded in paraffin. The sample is sectioned and stained with HE to evaluate the condition of the implanted region. Osteogenesis is not observed in any of the composite materials using the biocompatible scaffolds made of gelatin, collagen and hydroxyapatite, respectively when composite materials using mesenchymal stem cells and a biocompatible scaffold is implanted. Slight osteogenesis is observed in all of the composite materials using the biocompatible scaffolds made of gelatin, collagen and hydroxyapatite, respectively, when composite materials using osteoblasts differentiated from mesenchymal stem cells and a biocompatible scaffold is implanted. This osteogenesis is compared to that induced by implantation of composite materials using chondrocytes having the potential for hypertrophy derived from costa/costal cartilage and a biocompatible scaffold (see Example 1) and from sterna and a biocompatible scaffold (see Example 2). These comparisons show that composite material using chondrocytes having the potential for hypertrophy and a biocompatible scaffold have superior osteogenetic ability to materials that use mesenchymal stem cells or osteoblasts differentiated from mesenchymal stem cells.

### Summary of Comparative Examples 3-5

Osteogenesis is not observed when composite materials including the chondrocytes without the potential for hypertrophy, derived from auricular cartilage, articular cartilage or costal cartilage, and a biocompatible scaffold are implanted subcutaneously into rats. In contrast, osteogenesis is slightly observed when composite materials including osteoblasts or osteoblasts differentiated from mesenchymal stem cells, and a biocompatible scaffold are implanted subcutaneously into rats. The observed osteogenesis is compared to that induced by the implantation of composite materials using chondrocytes having the potential for hypertrophy and a biocompatible scaffold. The comparison showed that composite materials using chondrocytes having the potential for hypertrophy and a biocompatible scaffold have superior osteogenetic ability to composite materials that use osteoblasts, or osteoblasts differentiated from mesenchymal stem cells.

### Example 3

### Effect of Implantation of a Composite Material Using Chondrocytes having the Potential for Hypertrophy Derived from Costa/Costal Cartilage and a Biocompatible Scaffold, into a Region of Bone Deficient

(Preparation and Identification of Chondrocytes having the Potential for Hypertrophy from Costa/Costal Cartilage)

A composite material using chondrocytes having the potential for hypertrophy and a biocompatible scaffold was prepared by a method as described in Example 1.

(Production of Region of Bone Deficient)

Male rats (Wistar) were anesthetized. Skin in the femoral ortibial region was ablated and soft tissue retracted to one side to expose a femoral or tibial region, or the scalp was ablated to a septically expose the skull. A trephine bar or disc was attached to a dental trephine and used to make perforated or dissecting bone deficiencies at a femoral or tibial region, or in the skull. The composite material prepared above was implanted into the newly-made bone deficient region. Four or twelve weeks after implantation, the rats were sacrificed and the implanted region removed, fixed with 10% neutral buffered formalin, and embedded in paraffin. The sample was sectioned and stained with hematoxylin and eosin (HE) to evaluate the condition of the implanted region. Osteogenesis was observed in all of the composite materials using a biocompatible scaffold made of gelatin, collagen and hydroxyapatite, respectively.

### Comparative Example 6A

### Effect of Implantation of a Pellet of Chondrocytes having the Potential for Hypertrophy Derived from Costa/Costal Cartilage into a Bone Deficient Region

(Preparation of a Pellet of Chondrocytes having the Potential for Hypertrophy Derived from Costa/Costal Cartilage)

A pellet of chondrocytes having the potential for hypertrophy is prepared by a method as described in Comparative Example 1A, and implanted into a region of bone deficit. Four or twelve weeks after implantation, the condition of the implanted region is evaluated. Slight osteogenesis is observed when the pellet of chondrocytes having the potential for hypertrophy from costa/costal cartilage is implanted into the bone deficient region, however, the area is significantly smaller compared to when composite material using the chondrocyte having the potential for hypertrophy from costa/costal cartilage and a biocompatible scaffold is implanted into the bone deficient region (see, Example 3).

### Comparative Example 6B

### Effect of Implantation of Chondrocytes having the Potential for Hypertrophy Derived from Costa/Costal Cartilage alone into a Bone Deficient Region

Chondrocytes having the potential for hypertrophy obtained by a method as described in Example 1 are implanted alone into a bone deficient region in rats. No osteogenesis is observed when the chondrocytes having the potential for hypertrophy are implanted alone.

### Comparative Example 6C

### Effect of Implantation of Hydroxyapatite Alone into a Bone Deficient Region

Using a method as described in Example 3, hydroxyapatite scaffolds were implanted alone into a bone deficient region in rats. Sligh to steogenesis was observed around the implant when hydroxyapatite was implanted alone.

### Example 4

### Effect of Implantation of a Composite Material Using Chondrocytes having the Potential for Hypertrophy Derived from Sterna and a Biocompatible Scaffold into a Bone Deficient Region

The composite materials using chondrocytes having the potential for hypertrophy derived from sterna and a biocompatible scaffold, obtained by a method as described in Example 2, are implanted into a bone deficient region in rats. Four or twelve weeks after implantation, osteogenesis is observed in all of the composite materials using the biocompatible scaffolds made of gelatin, collagen and hydroxyapatite, respectively.

### Comparative Example 7A

### Effect of Implantation of a Pellet of Chondrocytes having the Potential for Hypertrophy Derived from Sterna into a Bone Deficient Region

Pellets of chondrocytes having the potential for hypertrophy are prepared by a method as described in Comparative Example 2A, and implanted into a bone deficient region in rats. Four or twelve weeks after implantation, the condition of the implanted region is evaluated. Slight osteogenesis is observed when a pellet of chondrocytes having the potential for hypertrophy from sterna is implanted into a bone deficient region, however, the area is significantly smaller compared to when a composite material using the chondrocyte having the potential for hypertrophy from sterna and a biocompatible scaffold is implanted into a bone deficient region (see, Example 4).

### Comparative Example 7B

### Effect of Implantation of Chondrocytes having the Potential for Hypertrophy Derived from Sterna Alone into a Bone Deficient Region

Chondrocytes having the potential for hypertrophy obtained by a method as described in Example 2 are implanted alone into a bone deficient region in rats. No osteogenesis is observed when chondrocytes having the potential for hypertrophy are implanted alone.

### Comparative Example 8A

### Effect of Implantation of a Composite Material Using Chondrocytes without the Potential for Hypertrophy Derived from Auricular Cartilage and a Biocompatible Scaffold into a Bone Deficient Region

The composite materials using chondrocytes without the potential for hypertrophy derived from auricular cartilage and a biocompatible scaffold, obtained in Comparative Example 3A, are implanted into a bone deficient region in rats. Four or twelve weeks after implantation, osteogenesis is not observed on the composite materials using the scaffolds made of either gelatin or collagen, and is only slightly observed around the implanted composite materials using a scaffold made of hydroxyapatite.

### Comparative Example 8B

### Effect of Implantation of a Composite Material Using Chondrocytes without the Potential for Hypertrophy Derived from Articular Cartilage and a Biocompatible Scaffold into a Bone Deficient Region

The composite materials using chondrocytes without the potential for hypertrophy derived from auricular cartilage and a biocompatible scaffold, obtained in Comparative Example 3B, are implanted into a bone deficient region in rats. Four or twelve weeks after implantation, osteogenesis is not observed in the composite materials using the biocompatible scaffolds made of gelatin or collagen, and only slightly observed around the implanted composite materials using the biocompatible scaffolds made of hydroxyapatite.

### Comparative Example 8C

### Effect of Implantation of a Composite Material Using Resting Cartilage Cells Derived from Costal Cartilage and a Biocompatible Scaffold into a Bone Deficient Region

The composite materials using chondrocytes without the potential for hypertrophy derived from costal cartilage and the biocompatible scaffold, obtained in Comparative Example 3C, are implanted into a bone deficient region in rats. Four or twelve weeks after implantation, osteogenesis is not observed in the composite materials using the biocompatible scaffolds made of gelatin or collagen, and only slightly observed around the implanted composite materials using the biocompatible scaffolds made of hydroxyapatite.

### Comparative Example 9

### Effect of Implantation of a Composite Material Using Osteoblasts and a Biocompatible Scaffold into a Bone Deficient Region

The composite materials using osteoblasts and the biocompatible scaffold, obtained in Comparative Example 4, are implanted into a bone deficient region in rats. Four or twelve weeks after implantation, osteogenesis is slightly observed in all the composite materials using biocompatible scaffolds made of gelatin, collagen and hydroxyapatite, respectively. The observed osteogenesis is compared to that induced by implantation of composite materials using chondrocytes having the potential for hypertrophy derived from costa/costal cartilage and a biocompatible scaffold (see Example 3) and from sterna and a biocompatible scaffold (see Example 4). These comparisons show that composite material using chondrocytes having the potential for hypertrophy and a biocompatible scaffold have superior osteogenetic ability to composite materials using osteoblasts.

### Comparative Example 10

### Comparison of the Rate and Quantity of Osteogenesis on Transplantation of a Composite Material Using Mesenchymal Stem Cells or Osteoblasts Derived from Mesenchymal Stem Cells and a Biocompatible Scaffold into a Bone Deficient Region

The composite materials using mesenchymal stem cells and the biocompatible scaffold, obtained in Comparative Example 5, or a composite materials using osteoblasts differentiated from mesenchymal stem cells and a biocompatible scaffold, are implanted into a bone deficient region in rats. Four or twelve weeks after the implantation of the composite material using mesenchymal stem cell and a biocompatible scaffold, slight osteogenesis is observed in all of the composite materials using the biocompatible scaffolds made of gelatin, collagen, and hydroxyapatite, respectively. When the composite material using osteoblasts differentiated from mesenchymal stem cells and a biocompatible scaffold is implanted, osteogenesis is slightly observed in all of the composite materials using the biocompatible scaffolds made of gelatin, collagen and hydroxyapatite, respectively. This osteogenesis is compared to that induced by implantation of composite materials using chondrocytes having the potential for hypertrophy derived from costa/costal cartilage and a biocompatible scaffold (see Example 3) and from sterna and a biocompatible scaffold (see Example 4). These comparisons show that composite material using chondrocytes having the potential for hypertrophy and a biocompatible scaffold have superior osteogenetic ability to materials that use mesenchymal stem cells or osteoblasts differentiated from mesenchymal stem cells.

### Summary of Examples 3-4 and Comparative Examples 6A-10

Osteogenesis observed after implantation of the composite material including chondrocytes having the potential for hypertrophy and a biocompatible scaffold into bone deficient regions in rats, is greater than that observed after implantation of the pelletized chondrocytes having the potential for hypertrophy. On the other hand, osteogenesis is not observed when chondrocytes having the potential for hypertrophy are implanted alone. When hydroxyapatite is implanted alone, osteogenesis is only slightly induced around the implant. These results mirror the comparative results obtained from subcutaneous testing of the composite material and single material (Examples 1-2 and Comparative Examples 1A-2B). Additionally, either in implantation of a composite materials using chondrocytes without the potential for hypertrophy derived from auricular cartilage, articular cartilage or resting cartilage and biocompatible scaffolds into bone deficient regions, or of a composite materials using osteoblasts, osteoblasts derived from mesenchymal stem cells or mesenchymal stem cells and biocompatible scaffolds into bone deficient regions, slight osteogenesis is observed in the bone deficient regions.

### Example 5

### The Rate and Quantity of Osteogenesis Induced by Implantation of a Composite Material Using Chondrocytes having the Potential for Hypertrophy and a Biocompatible Scaffold into a Bone Deficient Region

(Production of a Bone Deficient Region and Measuring the Rate and Quantity of Osteogenesis)

The rate and quantity of osteogenesis was measured after the composite material of Example 1 in which osteogenesis was observed, was implanted into a bone deficient region. The rate and quantity of osteogenesis was measured using micro CT (SkyScan 1172, Toyo Technica). Hydroxyapatite was used as a biocompatible scaffold. The composite material using chondrocytes having the potential for hypertrophy and the hydroxyapatite, obtained by a method as described in Example 1 (using a discoid hydroxyapatite of 4 mm in diameter×1 mm in thickness) was implanted into the bone deficient region (a punched lesion of 4 mm in diameter). The bone deficient region was made by a method described in Example 3. Four or twelve weeks after implantation, the implanted region was removed and measured with micro CT (65 kV-154 μA, 80 kV-125 μA, or 100 kV-100 μA; Al or Ti filter; rotation angle 0.4°).

(The Rate and Quantity of Osteogenesis)

As shown in Table 2, four weeks after implantation of composite material using chondrocytes having the potential for hypertrophy and hydroxyapatite, into the bone deficient region, 5.78 mm³ of bone was generated. The percentage of the neogenic bone to total bone was 45.90%.

### Comparative Example 11

### The Rate and Quantity of Osteogenesis Induced by Implantation of a Composite Material Using Resting Cartilage Cells without the Potential for Hypertrophy and a Biocompatible Scaffold into a Bone Deficient Region

(Producing a Bone Deficient Region and Measuring the Rate and Quantity of Osteogenesis)

Hydroxyapatite was used as a biocompatible scaffold. The composite material using chondrocytes without the potential for hypertrophy and the hydroxyapatite, obtained by a method as described in Example 3C (using a discoid hydroxyapatite of 4 mm in diameter×1 mm in thickness) was implanted into the bone deficient region (a lesion of 4 mm in diameter). The bone deficient region was made by a method as described in Example 3.

(The Rate and Quantity of Osteogenesis)

As shown in Table 2, four weeks after implantation of the composite material using chondrocytes without the potential for hypertrophy and hydroxyapatite into the bone deficient region, 2.74 mm³ of bone was generated. The percentage of the neogenic bone to total bone was 24.16%. The rate and quantity of osteogenesis was less than the composite material using chondrocytes having the potential for hypertrophy and hydroxyapatite, which is described in Example 5.

### Comparative Example 12

### The Rate and Quantity of Osteogenesis Induced by Implantation of a Biocompatible Scaffold Alone into a Bone Deficient Region

(Production of a Bone Deficient Region and Measuring the Rate and Quantity of Osteogenesis)

Hydroxyapatite was used as a biocompatible scaffold. The hydroxyapatite (using a discoid hydroxyapatite of 4 mm in diameter×1 mm in thickness) was implanted into the bone deficient region (a lesion of 4 mm in diameter). The bone deficient region was made by a method as described in Example 3. Four weeks after implantation, the implanted region was removed and measured with micro CT (65 kV-154 μA, 80 kV-125 μA, or 100 kV-100 μA; Al or Ti filter; rotation angle 0.4°).

(The Rate and Quantity of Osteogenesis)

As shown in Table 2, four weeks after implantation of hydroxyapatite alone into the bone deficient region, 2.7.2 mm³ of bone was generated. The percentage of the neogenic bone to total bone was 29.48%. The rate and quantity of osteogenesis was less than that of the composite material using chondrocytes having the potential for hypertrophy and hydroxyapatite, which is described in Example 5.

### Summary of Example 5 and Comparative Examples 11 and 12

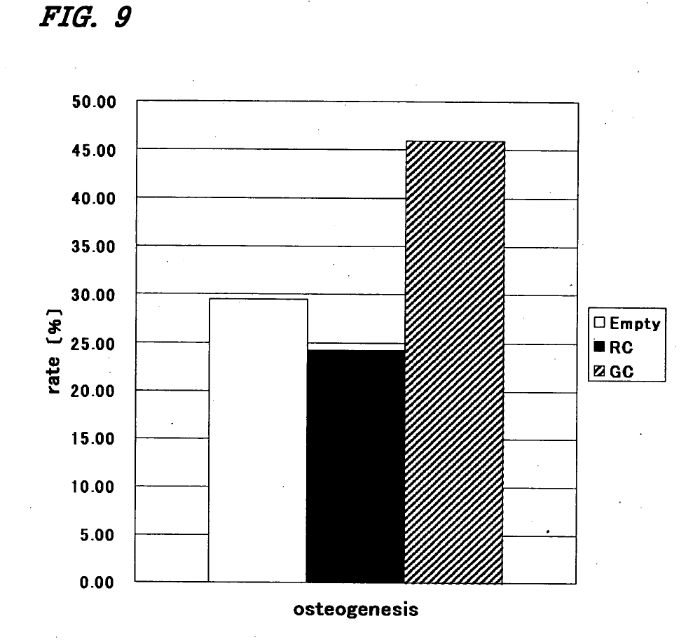

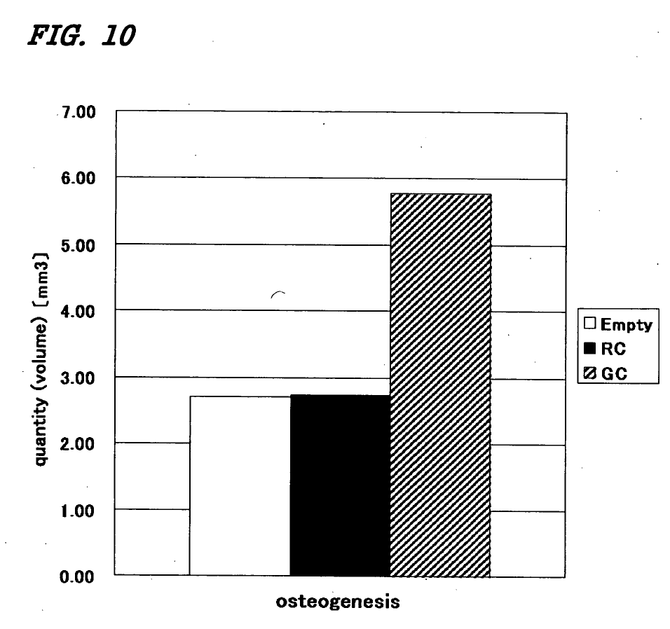

The rate and quantity of osteogenesis observed after implantation of composite material using chondrocytes having the potential for hypertrophy and hydroxyapatite into the bone deficient region was greater than that of the composite material using chondrocytes without the potential for hypertrophy and hydroxyapatite (see, FIGS. 9-10 and Table 2). When the composite material using chondrocytes having the potential for hypertrophy and hydroxyapatite was implanted into the bone deficient region, the rate and quantity of osteogenesis was greater than that that observed after implantation of hydroxyapatite alone (see, FIGS. 9-10 and Table 2). There is no difference between the rate and quantity of osteogenesis after the implantation of the composite material using chondrocytes without the potential for hypertrophy and the hydroxyapatite, and that after implantation hydroxyapatite alone, into a bone deficient region (see, FIGS. 9-10 and Table 2).

TABLE 2

| | Volume (mm³) | | | | Rate (%) | | | |
|---|---|---|---|---|---|---|---|---|
| | | neo- | | | | neo- | | |
| | | genic | | | | genic | | |
| | apatite | bone | void | total | apatite | bone | void | total |
| Empty | 0.94 | 2.72 | 5.56 | 9.21 | 10.23 | 29.48 | 60.29 | 100.00 |
| RC | 0.91 | 2.74 | 7.68 | 11.33 | 8.02 | 24.16 | 67.82 | 100.00 |
| GC | 1.50 | 5.78 | 5.32 | 12.60 | 11.89 | 45.90 | 42.20 | 100.00 |

Empty: the quantity (volume) and rate of osteogenesis 4 weeks after implantation of hydroxyapatite alone into a bone deficient region in rats' skulls.

GC: the quantity (volume) and rate of osteogenesis 4 weeks after implantation of a composite material using chondrocytes having the potential for hypertrophy (growth cartilage cell obtained from costa/costal cartilage)and hydroxyapatite into a bone deficient region in rats' skulls.

RC: the quantity (volume) and rate of osteogenesis 4 weeks after implantation of a composite material using chondrocytes without the potential for hypertrophy (resting cartilage cells obtained from costal cartilage) and hydroxyapatite into a bone deficient region in rats' skulls.

### Comparative Example 13

### The Rate and Quantity of Osteogenesis after Implantation of a Composite Material Using Osteoblasts and a Biocompatible Scaffold into a Bone Deficient Region

(Production of a Bone Deficient Region and Measuring the Rate and Quantity of Osteogenesis)

Hydroxyapatite is used as a biocompatible scaffold. The composite material using osteoblasts and hydroxyapatite, obtained by a method as described in Comparative Example 4 (using a discoid hydroxyapatite of 4 mm in diameter×1 mm in thickness) is implanted into a bone deficient region (a lesion of 4 mm in diameter). The bone deficient region is made by a method as described in Example 3. Four or twelve weeks after implantation, the implanted region is removed and measured with micro CT (65 kV-154 μA, 80 kV-125 μA, or 100 kV-100 μA; Al or Ti filter; rotation angle 0.4°).

(The Rate and Quantity of Osteogenesis)

Four or twelve weeks after the implantation of the composite material using osteoblasts and hydroxyapatite into a bone deficient region, bone is slightly generated. It is observed that the percentage of neogenic bone to total bone is low, and that the rate and quantity of osteogenesis is less than the composite material using chondrocytes having the potential for hypertrophy and hydroxyapatite, which is described in Example 5.

### Comparative Example 14

### The Rate and Quantity of Osteogenesis Induced by the Implantation of a Composite Material Using Mesenchymal Stem Cells or Osteoblasts Differentiated from Mesenchymal Stem Cells and a Biocompatible Scaffold into a Bone Deficient Region)

(Production of a Bone Deficient Region and Measuring the Rate and Quantity of Osteogenesis)

Hydroxyapatite is used as a biocompatible scaffold. The composite material using mesenchymal stem cells or an osteoblast differentiated from mesenchymal stem cells and hydroxyapatite, obtained by a method as described in Comparative Example 5 (using discoid hydroxyapatite of 4 mm in diameter×1 mm in thickness) is implanted into a bone deficient region (a lesion of 4 mm in diameter). The bone deficient region is made by a method as described in Example 3. Four or twelve weeks after implantation, the implanted region is removed and measured with micro CT (65 kV-154 μA, 80 kV-125 μA, or 100 kV-100 μA; Al or Ti filter; rotation angle 0.4°).

(The Rate and Quantity of Osteogenesis)

Four or twelve weeks after the implantation of the composite material using mesenchymal stem cells or osteoblasts differentiated from mesenchymal stem cell and hydroxyapatite into the bone deficient region, bone is slightly generated. It is observed that the percentage of the neogenic bone to total bone is low, and that the rate and quantity of osteogenesis is less than the composite material using chondrocytes having the potential for hypertrophy and hydroxyapatite, which is described in Example 5.

### Example 6

### Effect of Culture Medium on a Composite Material Using Chondrocytes having the Potential for Hypertrophy and a Biocompatible Scaffold

(Preparation and Identification of Chondrocytes having the Potential for Hypertrophy Costa/Costal Cartilage)

The composite material using chondrocytes having the potential for hypertrophy and a biocompatible scaffold was prepared using a method as described in Example 1.

(Producing a Composite Material Using Chondrocytes having the Potential for Hypertrophy Obtained from Costa/Costal Cartilage and a Biocompatible Scaffold)

MEM growth medium was added to the chondrocytes having the potential for hypertrophy obtained from Example 1, to a final concentration of 1×10⁶ cells/ml. This cell suspension was inoculated evenly to gelatin, collagen and hydroxyapatite scaffolds, respectively, and incubated in a 5% CO₂ incubator at 37° C. for 1 week.

These culture are implanted into rats subcutaneously. Four weeks after the implantation, the rats are sacrificed and the implanted region removed, fixed with 10% neutral buffered formalin, and embedded in paraffin. The samples are sectioned and stained with HE to evaluate the condition of the implanted region. Osteogenesis is observed in all of the composite materials using the biocompatible scaffolds made of gelatin, collagen and hydroxyapatite, respectively.

### Example 7

### Effect of Subcutaneous Implantation of a Composite Material Using Chondrocytes having the Potential for Hypertrophy Derived from a Human and a Biocompatible Scaffold

(Preparation of Chondrocytes having the Potential for Hypertrophy from a Human)

Chondrocytes having the potential for hypertrophy derived from human tissue such as polymeliac, tumor or donated cartilage tissue is obtained from organizations utilizing human tissue sources (Japanese organizations such as the Health Science Research Resources Bank, RIKEN Bioresource center, JCRB Cellbank of National Institute of Biomedical Innovation, and Institute of Development, Aging and Cancer in Tohoku university; non-Japanese organizations such as International Institute for the Advancement of Medicine (IIAM) and American Type Culture Collection (ATCC); and commercial sources such as Dainippon Sumitomo Pharmaceutical, Sanko Junyaku, TOYOBO, Cambrex, and Osiris). Obtained cells are inoculated to HamF12 growth medium.

(Identification of Chondrocytes having the Potential for Hypertrophy)

Using a method as described in Example 1, it is determinated that the cells obtained above are chondrocytes having the potential for hypertrophy.

(Producing a Composite Material Using Chondrocytes having the Potential for Hypertrophy Derived from a Human and a Biocompatible Scaffold)

Using the chondrocytes having the potential for hypertrophy obtained in the present Example, a composite material is prepared by a method as described in Example 1, and implanted into immunodeficient animals such as nude mice or nude rats subcutaneously. Four weeks after implantation, the animals are sacrificed and the implanted region removed, fixed with 10% neutral buffered formalin, and embedded in paraffin. The sample is sectioned and stained with HE to evaluate the condition of the implanted region. Osteogenesis is observed in all of composite materials using the biocompatible scaffolds made of gelatin, collagen and hydroxyapatite, respectively.

(Effect of Implantation of a Composite Material Using Chondrocytes having the Potential for Hypertrophy and a Biocompatible Scaffold into a Bone Deficient Region)

The bone deficient region is made by a method as described in Example 3. The composite material using chondrocytes having the potential for hypertrophy obtained in the present Example and a biocompatible scaffold is implanted into the bone deficient region. Four or twelve weeks after implantation, osteogenesis is observed in all of the composite materials using the biocompatible scaffold made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using chondrocytes having the potential for hypertrophy and hydroxyapatite into the bone deficient region, neogenesis of bone is observed.

### Comparative Example 15A

### Effect of Subcutaneous Implantation of a Composite Material Using Chondrocytes without the Potential for Hypertrophy Derived from a Human and a Biocompatible Scaffold

(Preparation and Identification of the Chondrocytes without the Potential for Hypertrophy from a Human)

Chondrocytes without the potential for hypertrophy derived from human tissue such as polymeliac, tumor or donated cartilage tissue is obtained from organizations utilizing human tissue sources (Japanese organizations such as the Health Science Research Resources Bank, RIKEN Bioresource center, JCRB Cellbank of National Institute of Biomedical Innovation, and Institute of Development, Aging and Cancer in Tohoku university; non-Japanese organizations such as IIAM and ATCC; and commercial sources such as Dainippon Sumitomo Pharmaceutical, Sanko Junyaku, TOYOBO, Cambrex, and Osiris). The cells are inoculated into HamF12 growth medium. Using a method as described in Example 1, it is determined if the prepared cells are chondrocytes without the potential for hypertrophy.

(Producing a Composite Material Using Chondrocytes without the Potential for Hypertrophy from a Human)

Using the chondrocytes without the potential for hypertrophy obtained in the present Example, a composite material is prepared by a method as described in Example 1, and implanted into immunodeficient animals such as nude mice or nude rats subcutaneously. Four weeks after implantation, the animals are sacrificed and the implanted region removed, fixed with 10% neutral buffered formalin, and embedded in paraffin. The sample is sectioned and stained with HE to evaluate the condition of the implanted region. Osteogenesis is not observed in any of composite materials using the biocompatible scaffolds made of gelatin, collagen and hydroxyapatite, respectively.

(Effect of Implantation of a Composite Material Using Chondrocytes without the Potential for Hypertrophy and a Biocompatible Scaffold into a Bone Deficient Region)

The bone deficient region is made by a method as described in Example 3. The composite material using chondrocytes without the potential for hypertrophy obtained in the present Example and a biocompatible scaffold is implanted into the bone deficient region. Four or twelve weeks after the implantation, osteogenesis is not observed in the composite materials using the biocompatible scaffold made of gelatin and collagen, and only slightly observed around the implanted composite material using the biocompatible scaffold made of hydroxyapatite.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using chondrocytes without the potential for hypertrophy and hydroxyapatite into the bone deficient region, slight neogenesis of bone is observed.

### Comparative Example 15B

### Effect of Subcutaneous Implantation of a Composite Material Using Osteoblasts Derived from a Human and a Biocompatible Scaffold

(Preparation and Identification of Osteoblast Derived from a Human)

Human osteoblasts are obtained from organizations utilizing human tissue sources (Japanese organizations such as the Health Science Research Resources Bank, RIKEN Bioresource center, JCRB Cellbank of National Institute of Biomedical Innovation, and Institute of Development, Aging and Cancer in Tohoku university; non-Japanese organizations such as IIAM and ATCC; and commercial sources such as Dainippon Sumitomo Pharmaceutical, Sanko Junyaku, TOYOBO, Cambrex, and Osiris). The cells are inoculated into MEM growth medium. Using a method as described in Example 4, the prepared cells are determined to be osteoblasts.

(Producing a Composite Material Using Osteoblast from a Human and a Biocompatible Scaffold)

Using the osteoblasts obtained in the present Example, a composite material is prepared by a method as described in Example 1, and subcutaneously implanted into immuno deficient animals such as nude mice or nude rats. Four weeks after implantation, the animals are sacrificed and the implanted region removed, fixed with 10% neutral buffered formalin, and embedded in paraffin. The sample is sectioned and stained with HE to evaluate the condition of the implanted region. Slight osteogenesis is observed in any of the composite materials using the biocompatible scaffolds made of gelatin, collagen and hydroxyapatite, respectively.

(Effect of Implantation of a Composite Material Using Osteoblasts and a Biocompatible Scaffold into a Bone Deficient Region)

The bone deficient region is made by a method as described in Example 3. The composite material using osteoblasts and a biocompatible scaffold obtained in the present Comparative Example, is implanted into the bone deficient region. Four or twelve weeks after implantation, slight osteogenesis is observed in all of the composite materials using the biocompatible scaffolds made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using osteoblasts and hydroxyapatite into the bone deficient region, slight neogenesis of bone is observed.

### Comparative Example 15C

### Effect of Subcutaneous Implantation of a Composite Material Using Mesenchymal Stem Cell or Osteoblasts Derived from Mesenchymal Stem Cells Derived from Human and a Biocompatible Scaffold

(Preparation and Identification of Mesenchymal Stem Cell Derived from a Human)

Human mesenchymal stem cells are obtained from organizations utilizing human tissue sources (Japanese organizations such as the Health Science Research Resources Bank, RIKEN Bioresource center, JCRB Cellbank of National Institute of Biomedical Innovation, and Institute of Development, Aging and Cancer in Tohoku university; non-Japanese organizations such as IIAM and ATCC; and commercial sources such as Dainippon Sumitomo Pharmaceutical, Sanko Junyaku, TOYOBO, Cambrex, and Osiris). The cells are inoculated into HamF12 growth medium.

(Producing a Composite Material Using Mesenchymal Stem Cell or Osteoblast from Mesenchymal Stem Cell Derived from Human, and a Biocompatible Scaffold)

A composite material is prepared using a method as described in Example 1, except for using mesenchymal stem cells obtained in the present Comparative Example and MEM growth medium. The composite material is further incubated in MEM differentiation medium, in 5% CO₂ at 37° C. for 2 weeks and the mesenchymal stem cell on or within the biocompatible scaffold differentiate to osteoblasts. The composite material using the differentiated osteoblast and hydroxyapatite is implanted subcutaneously into immunodeficient animals such as nude mice or nude rats. A composite material using a biocompatible scaffold and mesenchymal stem cells without undergoing the 2 weeks differentiation procedure, is also used for implantation. Four weeks after implantation, the animals are sacrificed and the implanted region removed, fixed with 10% neutral buffered formalin, and embedded in paraffin. The sample is sectioned and stained with HE to evaluate the condition of the implanted region. Osteogenesis is not observed in any of the composite materials using biocompatible scaffolds made of gelatin, collagen and hydroxyapatite, respectively, when composite materials using mesenchymal stem cells and a biocompatible scaffold are implanted subcutaneously. Slight osteogenesis is observed in all of the composite materials using the biocompatible scaffolds made of gelatin, collagen and hydroxyapatite, respectively, when the composite materials using osteoblasts differentiated from mesenchymal stem cells and a biocompatible scaffold are implanted.

(Effect of Implantation of a Composite Material Using Mesenchymal Stem Cells or Osteoblasts Derived from Mesenchymal Stem Cells Derived from Human, and a Biocompatible Scaffold into a Bone Deficient Region)

The bone deficient region is produced by a method as described in Example 3. The composite material using mesenchymal stem cells or osteoblasts derived from mesenchymal stem cells and a biocompatible scaffold, obtained in the present Comparative Example, is implanted into the bone deficient region. Four or twelve weeks after the implantation, slight osteogenesis is observed in all of the composite materials using the biocompatible scaffolds made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after implantation of the composite material using mesenchymal stem cells or osteoblasts derived from mesenchymal stem cells and hydroxyapatite into the bone deficient region, slight neogenesis of bone is observed.

### Example 8

### Effect of Subcutaneous Implantation of a Composite Material Using Chondrocytes having the Potential for Hypertrophy Derived from Murine Costa/Costal Cartilage and a Biocompatible Scaffold)

(Preparation and Identification of the Chondrocytes having the Potential for Hypertrophy Derived from Murine Costa/Costal Cartilage)

Mice are used as subjects. Chondrocytes having the potential for hypertrophy obtained in the present Example are prepared from the murine costa/costal cartilage using a method as described in Example 1. Prepared cells are identified as chondrocytes having the potential for hypertrophy using a method as described in Example 1.

(Producing a Composite Material Using Chondrocytes having the Potential for Hypertrophy Derived from Murine Costa/Costal Cartilage and a Biocompatible Scaffold)

A composite material is prepared using chondrocytes having the potential for hypertrophy obtained in the present Example by a method as described in Example 1, and implanted subcutaneously into mice. Four weeks after implantation, the mice are sacrificed and the implanted region removed, fixed with 10% neutral buffered formalin, and embedded in paraffin. The sample is sectioned and stained with HE to evaluate the condition of the implanted region. Osteogenesis is observed in all of the composite materials using the biocompatible scaffolds made of gelatin, collagen and hydroxyapatite, respectively. (Effect of Implantation of a Composite Material Using Chondrocytes having the Potential for Hypertrophy and a Biocompatible Scaffold into a Bone Deficient Region)

The bone deficient region is made by a method as described in Example 3. The composite material using chondrocytes having the potential for hypertrophy and a biocompatible scaffold, obtained in the present Example, is implanted into the bone deficient region. Four or twelve weeks after implantation, osteogenesis is observed in all of the composite materials using biocompatible scaffolds made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using chondrocytes having the potential for hypertrophy and hydroxyapatite into the bone deficient region, neogenesis of bone is observed.

### Comparative Example 16A

### Effect of Subcutaneous Implantation of a Composite Material Using Resting Cartilage Cells without the Potential for Hypertrophy Derived from Murine Costa/Costal Cartilage and a Biocompatible Scaffold

(Preparation and Identification of Resting Cartilage Cells from Murine Costal Cartilage)

Mice are used as subjects. Resting cartilage cells are prepared using a method as described in Comparative Example 3C. Prepared cells are identified as being resting cartilage cells without the potential for hypertrophy using a method as described in Comparative Example 3C.

(Producing a Composite Material Using Resting Cartilage Cells without the Potential for Hypertrophy Derived from Murine Costal Cartilage and a Biocompatible Scaffold)

A composite material is prepared using chondrocytes without the potential for hypertrophy obtained in the present Comparative Example by a method as described in Example 1, and subcutaneously implanted into mice. Four weeks after implantation, the mice are sacrificed and the implanted region removed, fixed with 10% neutral buffered formalin, and embedded in paraffin. The sample is sectioned and stained with HE to evaluate the condition of the implanted region. No osteogenesis is observed in any of the composite materials using the biocompatible scaffolds made of gelatin, collagen and hydroxyapatite, respectively.

(Effect of Implantation of a Composite Material Using Chondrocytes without the Potential for Hypertrophy and a Biocompatible Scaffold into a Bone Deficient Region)

The bone deficient region is made by a method as described in Example 3. The composite material using chondrocytes without the potential for hypertrophy and a biocompatible scaffold, obtained in the present Comparative Example, is implanted into the bone deficient region. Four or twelve weeks after implantation, osteogenesis is not observed in any of the composite materials using biocompatible scaffolds made of gelatin and collagen, and only slightly observed around the implanted composite materials using the biocompatible scaffold made of hydroxyapatite.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using chondrocytes without the potential for hypertrophy and hydroxyapatite into the bone deficient region, slight neogenesis of bone is observed.

### Comparative Example 16B

### Effect of Subcutaneous Implantation of a Composite Material Using Osteoblasts Derived from Mice and a Biocompatible Scaffold

(Preparation and Identification of Osteoblast Derived from Mice)

Mice are used as subjects. Osteoblasts are prepared from mice using a method as described in Comparative Example 4. The prepared cells are identified to be osteoblasts using a method as described in Comparative Example 4.

(Producing a Composite Material Using Osteoblasts Derived from Mice and a Biocompatible Scaffold)

A composite material is prepared using osteoblasts obtained in the present Comparative Example by a method as described in Example 1, and subcutaneously implanted into mice. Four weeks after the implantation, the mice are sacrificed and the implanted region removed, fixed with 10% neutral buffered formalin, and embedded in paraffin. The sample is sectioned and stained with HE to evaluate the condition of the implanted region. Slight osteogenesis is observed in all of the composite materials using the biocompatible scaffolds made of gelatin, collagen and hydroxyapatite, respectively.

(Effect of Implantation of a Composite Material Using Osteoblasts Derived from Mice and a Biocompatible Scaffold into a Bone Deficient Region)

The bone deficient region is made by a method as described in Example 3. The composite material using osteoblasts and a biocompatible scaffold, obtained in the present Comparative Example, is implanted into the bone deficient region. Four or twelve weeks after implantation, slight osteogenesis is observed in all of the composite materials using the biocompatible scaffold made of gelatin, collagen and hydroxyapatite, respectively.

(Measuring the Rate and Quantity of Osteogenesis in the bone Deficient Region)

The rate and quantity of osteogenesis in the implanted region is measured by a method as described in Example 5. Four or twelve weeks after the implantation of the composite material using osteoblasts and hydroxyapatite into the bone deficient region, slight neogenesis of bone is observed.

### Comparative Example 16C

### Effect of Subcutaneous Implantation of a Composite Material Using Mesenchymal Stem Cells or Osteoblasts Derived from Mesenchymal Stem Cells Derived from Mice and a biocompatible Scaffold

(Preparation and Identification of Mesenchymal Stem Cells Derived from Mice)

Mice are used as subjects. Mesenchymal stem cells are prepared from mice using a method as described in Comparative Example 5. The prepared cells are identified as mesenchymal stem cells using a method as described in Comparative Example 14.

(Producing a Composite Material Using Mesenchymal Stem Cells or Osteoblasts Derived from Mesenchymal Stem Cell Derived from Mice and a Biocompatible Scaffold)

A composite material is prepared using mesenchymal stem cells obtained in the present Comparative Example by a method as described in Example 1, except for using mesenchymal stem cells obtained in the present Comparative Example and MEM growth medium. The composite material is further incubated in MEM differentiation medium, in 5% CO₂ at 37° C. for 2 weeks, to differentiate the mesenchymal stem cells on or within the biocompatible scaffold in to osteoblasts. The composite material using the differentiated osteoblasts and hydroxyapatite is subcutaneously implanted into mice. A composite material using a biocompatible scaffold and mesenchymal stem cells without undergoing the 2 week differentiation procedure, is also used for the implantation. Four weeks after implantation, the mice are sacrificed and the implanted region removed, fixed with 10% neutral buffered formalin, and embedded in paraffin. The sample is sectioned and stained with HE to evaluate the condition of the implanted region. After subcutaneous implantation of the composite material using mesenchymal stem cell and a biocompatible scaffold, no osteogenesis is observed in any of the composite materials using the biocompatible scaffolds made of gelatin, collagen and hydroxyapatite, respectively. After subcutaneous implantation of the composite material using osteoblasts differentiated from mesenchymal stem cells and a biocompatible scaffold, slight osteogenesis is observed in all of the composite materials using the biocompatible scaffolds made of gelatin, collagen and hydroxyapatite, respectively.

(Effect of Implantation of a Composite Material Using Mesenchymal Stem Cells or Osteoblasts Derived from Mesenchymal Stem Cells and a Biocompatible Scaffold into a Bone Deficient Region)

The bone deficient region is made by a method as described in Example 3. The composite material using mesenchymal stem cells or osteoblasts derived from mesenchymal stem cells and a biocompatible scaffold, obtained in the present Comparative Example, are implanted into the bone deficient region. Four or twelve weeks after implantation, slight osteogenesis is observed in all of the composite materials using the biocompatible scaffold made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using mesenchymal stem cells or osteoblasts derived from mesenchymal stem cells and hydroxyapatite into the bone deficient region, slight neogenesis of bone is observed.

### Example 9

### Effect of Subcutaneous Implantation of a Composite Material Using Chondrocytes having the Potential for Hypertrophy Derived from Rabbit Costa/Costal Cartilage and a Biocompatible Scaffold)

(Preparation of Chondrocytes having the Potential for Hypertrophy from Costa/Costal Cartilage)

Rabbits are used as subjects. Chondrocytes having the potential for hypertrophy are prepared from rabbit costa/costal cartilage using a method as described in Example 1.

(Identification of Chondrocytes having the Potential for Hypertrophy)

The prepared cells are identified as chondrocytes having the potential for hypertrophy using a method as described in Example 1.

(Producing a Composite Material Using Chondrocytes having the Potential for Hypertrophy from Rabbit Costa/Costal Cartilage and a Biocompatible Scaffold)

A composite material is prepared using chondrocytes having the potential for hypertrophy obtained in the present Example by a method as described in Example 1, and implanted subcutaneously into rabbits. Four weeks after implantation, the rabbits are sacrificed and the implanted region removed, fixed with 10% neutral buffered formalin, and embedded in paraffin. The sample is sectioned and stained with HE to evaluate the condition of the implanted region. Osteogenesis is observed in all of the composite materials using the biocompatible scaffolds made of gelatin, collagen and hydroxyapatite, respectively.

(Effect of Implantation of a Composite Material Using Chondrocytes having the Potential for Hypertrophy and a Biocompatible Scaffold into Bone Deficient Region)

The bone deficient region is made by a method as described in Example 3. The composite material using chondrocytes having the potential for hypertrophy and a biocompatible scaffold, obtained in the present Example, is implanted into the bone deficient region. Four or twelve weeks after implantation, osteogenesis is observed in all of the composite materials using the biocompatible scaffold made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using chondrocytes having the potential for hypertrophy and hydroxyapatite into the bone deficient region, neogenesis of bone is observed.

### Comparative Example 17A

### Effect of Subcutaneous Implantation of a Composite Material Using Resting Cartilage Cells without the Potential for Hypertrophy Derived from Rabbit Costal Cartilage and a Biocompatible Scaffold)

(Preparation and Identification of Resting Cartilage Cells without the Potential for Hypertrophy from Rabbit Costal Cartilage)

Rabbits are used as subjects. Resting cartilage cells are prepared using a method as described in Comparative Example 3C. The prepared cells are identified as resting cartilage cells without the potential for hypertrophy, using a method as described in Comparative Example 3C.

(Producing a Composite Material Using Resting Cartilage Cells without the Potential for Hypertrophy Derived from Rabbit Costal Cartilage and a Biocompatible Scaffold)

A composite material is prepared using the resting cartilage cells without the potential for hypertrophy obtained in the present Example by a method as described in Example 1, and subcutaneously implanted into rabbits. Four weeks after the implantation, the rabbits are sacrificed and the implanted region removed, fixed with 10% neutral buffered formalin, and embedded in paraffin. The sample is sectioned and stained with HE to evaluate the condition of the implanted region. No osteogenesis is observed in any of the composite materials using the biocompatible scaffolds made of gelatin, collagen and hydroxyapatite, respectively.

(Effect of Implantation of a Composite Material Using Resting Cartilage Cells without the Potential for Hypertrophy and a Biocompatible Scaffold into a Bone Deficient Region)

The bone deficient region is made by a method as described in Example 3. The composite material using resting cartilage cells without the potential for hypertrophy and a biocompatible scaffold, obtained in the present Comparative Example, is implanted into the bone deficient region. Four or twelve weeks after implantation, osteogenesis is not observed in any of the composite materials using the biocompatible scaffold made of gelatin and collagen, and only slightly observed around the implanted composite materials using the biocompatible scaffold made of hydroxyapatite.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using chondrocytes without the potential for hypertrophy and hydroxyapatite into the bone deficient region, slight neogenesis of bone is observed.

### Comparative Example 17B

### Effect of Subcutaneous Implantation of a Composite Material Using Osteoblasts Derived from a Rabbit and a Biocompatible Scaffold

(Preparation and Identification of Osteoblasts Derived from a Rabbit)

Rabbits are used as subjects. Osteoblasts are prepared from rabbits using a method as described in Comparative Example 4. The prepared cells are identified as osteoblasts using a method as described in Comparative Example 4.

(Producing a Composite Material Using Osteoblasts Derived from a Rabbit and a Biocompatible Scaffold)

A composite material is prepared using osteoblasts obtained in the present Comparative Example by a method as described in Example 1, and subcutaneously implanted into rabbits. Four weeks after implantation, the rabbits are sacrificed and the implanted region removed, fixed with 10% neutral buffered formalin, and embedded in paraffin. The sample is sectioned and stained with HE to evaluate the condition of the implanted region. Slight osteogenesis is observed in all of the composite materials using biocompatible scaffolds made of gelatin, collagen and hydroxyapatite, respectively.

(Effect of Implantation of a Composite Material Using Osteoblast and a Biocompatible Scaffold into a Bone Deficient Region)

The bone deficient region is made by a method as described in Example 3. The composite material using osteoblasts and a biocompatible scaffold, obtained in the present Comparative Example, is implanted into the bone deficient region. Four or twelve weeks after implantation, slight osteogenesis is observed in all of the composite materials using a biocompatible scaffold made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using osteoblasts and hydroxyapatite into the bone deficient region, slight neogenesis of bone is observed.

### Comparative Example 17C

### Effect of Subcutaneous Implantation of a Composite Material Using Mesenchymal Stem Cells or Osteoblasts Derived from Mesenchymal Stem Cells Derived from a Rabbit and a Biocompatible Scaffold)

(Preparation and Identification of Mesenchymal Stem Cells Derived from a Rabbit)

Rabbits are used as subjects. Mesenchymal stem cells are prepared using a method as described in Comparative Example 5. The prepared cells are identified as mesenchymal stem cells using a method as described in Comparative Example 5.

(Producing a Composite Material Using Mesenchymal Stem Cells or Osteoblasts Derived from Mesenchymal Stem Cells Derived from Rabbits and a Biocompatible Scaffold)

A composite material is prepared using mesenchymal stem cells obtained in the present Comparative Example by a method as described in Example 1, except for using mesenchymal stem cells obtained in the present Comparative Example and MEM growth medium. The composite material is further incubated in MEM differentiation medium, in 5% CO₂ at 37° C. for 2 weeks, to differentiate the mesenchymal stem cell on or within the biocompatible scaffold into osteoblasts. The composite material using the differentiated osteoblasts and hydroxyapatite is subcutaneously implanted into rabbits. A composite material using a biocompatible scaffold and mesenchymal stem cells without undergoing the 2 week differentiation procedure, is also used for implantation. Four weeks after implantation, the rabbits are sacrificed and the implanted region removed, fixed with 10% neutral buffered formalin, and embedded in paraffin. The sample is sectioned and stained with HE to evaluate the condition of the implanted region. After subcutaneous implantation of the composite material using mesenchymal stem cells and a biocompatible scaffold, no osteogenesis is observed in any of the composite materials using the biocompatible scaffolds made of gelatin, collagen and hydroxyapatite, respectively. After subcutaneous implantation of the composite material using osteoblasts differentiated from mesenchymal stem cells and a biocompatible scaffold, osteogenesis is slightly observed in all of the composite materials using the biocompatible scaffolds made of gelatin, collagen and hydroxyapatite, respectively.

(Effect of Implantation of a Composite Material Using Mesenchymal Stem Cells or Osteoblasts Derived from Mesenchymal Stem Cells and a Biocompatible Scaffold into Bone Deficient Region)

The bone deficient region is made by a method as described in Example 3. The composite material using mesenchymal stem cells or osteoblasts derived from mesenchymal stem cells and a biocompatible scaffold, obtained in the present Comparative Example, is implanted into the bone deficient region. Four or twelve weeks after implantation, slight osteogenesis is observed in all of the composite materials using the biocompatible scaffold made of gelatin, collagen and hydroxyapatite, respectively.

(Measuring the Rate and Quantity of Osteogenesis in the Bone Deficient Region)

The rate and quantity of osteogenesis in the implanted region is measured by a method as described in Example 5. Four or twelve weeks after the implantation of the composite material using mesenchymal stem cells or osteoblasts derived from mesenchymal stem cells and hydroxyapatite into the bone deficient region, slight neogenesis of bone is observed.

As discussed above, the present invention has been illustrated by preferred embodiments of the invention, however, the present invention should not be considered to be limited by such embodiments. It is appreciated that the present invention should be limited only by the scope of claims. It is understood that those skilled in the art can perform equivalents of the invention according to the description of the invention or the common technical knowledge within the art. It is also understood that the contents of patents, patent application and literatures cited herein should be incorporated herein by reference, as if their contents are specifically described herein.

### INDUSTRIAL APPLICABILITY

The present invention provides a composite material comprising chondrocytes having the potential for hypertrophy and a scaffold that is biocompatible with the biological organism, having a property that causes the unexpected progression of osteogenesis as a combination of cells and scaffolds, and thereby making it possible to use the combination of chondrocytes having the potential for hypertrophy and a scaffold, that was conventionally not considered to be practical at a practical level, and to treat, regions which previously had a poor prognosis after implantation of prior art artificial materials.

What is claimed is:

1. A composite material for enhancing or inducing osteogenesis in a biological organism, comprising:
A) a chondrocyte having the potential for hypertrophy, and
B) a scaffold that is biocompatible with the biological organism.

2. The composite material according to claim 1, wherein the osteogenesis is for repairing a defective region of bone.

3. The composite material according to claim 2, wherein the composite material is used to ameliorate osteogenesis in the defective region of the bone having a size that is incapable of being repaired by fixation alone.

4. The composite material according to claim 1, wherein the chondrocyte having the potential for hypertrophy is contained in a region which is selected from the group consisting of a surface, and a region within an internal pore, of the scaffold that is biocompatible with the biological organism.

5. The composite material according to claim 1, wherein the chondrocyte having the potential for hypertrophy expresses at least one marker selected from the group consisting of type X collagen, alkaline phosphatase, osteonectin, type II collagen, cartilage proteoglycan or components thereof, hyaluronic acid, type IX collagen, type XI collagen and chondromodulin.

6. The composite material according to claim 1, wherein the chondrocyte having the potential for hypertrophy is characterized by morphological hypertrophy.

7. The composite material according to claim 6, wherein the chondrocyte is determined to have the potential for hypertrophy by observing its significant proliferation by preparing a pellet of the cells by centrifugation of 5×10⁵ cells in culture medium, culturing the pellet for a pre-determined period, and comparing a size of the cells observed under a microscope before culture with that after culture.

8. The composite material according to claim 1, wherein the chondrocyte having the potential for hypertrophy is derived from a mammal.

9. The composite material according to claim 8, wherein the chondrocyte having the potential for hypertrophy is derived from a human, a mouse, a rat, a rabbit, a dog, a cat, or a horse.

10. The composite material according to claim 1, wherein the chondrocyte having the potential for hypertrophy is derived from an allogenic individual.

11. The composite material according to claim 1, wherein the chondrocyte having the potential for hypertrophy is derived from a heterologous individual.

12. The composite material according to claim 1, wherein the chondrocyte having the potential for hypertrophy is a cell obtained from a portion selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture and the cartilaginous part of bone proliferation phase.

13. The composite material according to claim 12, wherein the epiphysial line of the long bone is a region selected from the group consisting of femoris, tibia, fibula, humerus, ulna and radius.

14. The composite material according to claim 12, wherein the zone of proliferating cartilage of ossicle is a region selected from the group consisting of hand bones, foot bones and sterna.

15. The composite material according to claim 1, wherein the chondrocyte having the potential for hypertrophy is adjusted to a cell density of 1×10⁷ cells/ml to 1×10⁴ cells/ml.

16. The composite material according to claim 1, wherein the chondrocyte having the potential for hypertrophy is a cell cultured in a medium comprising one selected from the group consisting of Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb), and a composition thereof.

17. The composite material according to claim 16, wherein the medium includes a material that enhances the proliferation, differentiation or both of cells.

18. The composite material according to claim 16, wherein the medium includes at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF).

19. The composite material according to claim 1, wherein the scaffold that is biocompatible with the biological organism comprises a material selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon, and a combination thereof.

20. The composite material according to claim 19, wherein the scaffold that is biocompatible with the biological organism is comprised of calcium phosphate, gelatin, collagen or a combination thereof.

21. The composite material according to claim 20, wherein the scaffold that is biocompatible with the biological organism is comprised of hydroxyapatite.

22. A method of producing of a composite material for enhancing or inducing osteogenesis in a biological organism, comprising the steps of:
A) providing a chondrocyte having the potential for hypertrophy, and
B) culturing the chondrocyte having the potential for hypertrophy on a scaffold that is biocompatible with the biological organism.

23. The method according to claim 22, wherein step A) comprises providing the chondrocyte having the potential for hypertrophy, wherein the potential for hypertrophy is indentifed by the expression of at least one selected from, but not limited to the group consisting of type X collagen, alkaline phosphatase, osteonectin, type II collagen, cartilage proteoglycan or components thereof, hyaluronic acid, type IX collagen, type XI collagen, and chondromodulin, as a marker.

24. The method according to claim 22, wherein step A) comprises the steps of:
providing the chondrocyte having the potential for hypertrophy, wherein the potential for hypertrophy is indentifed using its hypertrophy as a marker;
preparing a pellet of the cells by centrifugation of 5×10⁵ cells in culture medium;
culturing the pellet for a pre-determined period;
comparing the size of the cells observed under a microscope before culture with that after culture; and
determining the chondrocyte as having the potential for hypertrophy when significant proliferation is observed.

25. The method according to claim 22, wherein the chondrocyte having the potential for hypertrophy is derived from a mammal.

26. The method according to claim 25, wherein the chondrocyte having the potential for hypertrophy is derived from a human, a mouse, a rat, a rabbit, a dog, a cat or a horse.

27. The method according to claim 22, wherein the chondrocyte having the potential for hypertrophy is a cell obtained from a portion selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture and the cartilaginous part of bone proliferation phase.

28. The method according to claim 27, wherein the epiphysial line of the long bone is a region selected from the group consisting of femoris, tibia, fibula, humerus, ulna and radius.

29. The method according to claim 27, wherein the zone of proliferating cartilage of ossicle is a region selected from the group consisting of hand bone, foot bone and the sterna.

30. The method according to claim 22, wherein the chondrocyte having the potential for hypertrophy is adjusted to a cell density of 1×10⁷ cells/ml to 1×10⁴ cells/ml.

31. The method according to claim 22, wherein step B) comprises culturing the chondrocyte having the potential for hypertrophy in a medium comprising one selected from the group consisting of Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb) and a combination thereof.

32. The method according to claim 22, wherein step B) comprises culturing the chondrocyte having the potential for hypertrophy in a medium including a substance that enhances the proliferation, differentiation or both of cells.

33. The method according to claim 22, wherein step B) comprises culturing the chondrocyte having the potential for hypertrophy in a medium including at least one component selected from the group consisting of transforming growth factor-beta (TGF-beta), bone morphogenetic factor (BMP), leukemia inhibitory factor (LIF), colony stimulating factor (CSF), ascorbic acid, dexamethasone, glycerophosphoric acid, insulin-like growth factor (IGF), fibroblast growth factor (FGF), platelet-rich plasma (PRP), platelet-derived growth factor (PDGF), and vascular endothelial growth factor (VEGF).

34. The method according to claim 22, wherein the scaffold that is biocompatibile with the biological organism includes a material selected from the group consisting of calcium phosphate, calcium carbonate, alumina, zirconia, apatite-wollastonite deposited glass, gelatin, collagen, chitin, fibrin, hyaluronic acid, silk, cellulose, dextran, polylactic acid, polyleucine, alginic acid, polyglycolic acid, methyl polymethacrylate, polycyanoacrylate, polyacrylonitrile, polyurethan, polypropylene, polyethylene, polyvinyl chloride, ethylene-vinyl acetate copolymer, nylon and a combination thereof.

35. The method according to claim 34, wherein the scaffold that is biocompatible with the biological organism is calcium phosphate, gelatin or collagen.

36. The method according to claim 35, wherein the scaffold that is biocompatible with the biological organism is hydroxyapatite.

37. The method according to claim 22, wherein the chondrocyte having the potential for hypertrophy is a cell cultured in a region selected from the group consisting of a surface and within an internal pore of the scaffold that is biocompatible with the biological organism, at 37° C. in the presence of 5-10% CO₂.

38. The method according to claim 22, wherein the step of culturing is performed for sufficient period such that the chondrocyte having the potential for hypertrophy is fixed on the scaffold that is biocompatible with the biological organism.

39. Use of a composite material to produce an implant or a bone repairing material for enhancing or inducing osteogenesis in a biological organism, the composite material comprising:
A) a chondrocyte having the potential for hypertrophy, and
B) a scaffold that is biocompatible with the biological organism.

40. A method of repairing a defective region of bone, comprising implanting a composite material including a chondrocyte having the potential for hypertrophy and a scaffold that is biocompatible a biological organism into the defective region of the bone.

41. The method according to claim 40, wherein the composite material is used to ameliorate osteogenesis in the defective region of the bone having a size that is incapable of being repaired by fixation alone.

42. A method of preparing of chondrocyte having the potential for hypertrophy, comprising the steps of obtaining cells from the processus xiphoideus junction located in the inferior portion of the corpus sterni.
